(19) United States
(12) Patent Application Publication
(10) Pub. No.: US 2025/0316328 A1
(43) Pub. Date: Oct. 9, 2025

(54) Method, System and Apparatus for Predicting PK Values of Antibodies

(71) Applicant: Sanofi, PARIS (FR)

(72) Inventors: José Antonio Amengual Rigo, Frankfurt am Main (DE); Joerg Birkenfeld, Frankfurt am Main (DE); Norbert Furtmann, Frankfurt am Main (DE)

(21) Appl. No.: 18/865,122

(22) PCT Filed: May 16, 2023
(86) PCT No.: PCT/EP2023/063077

§ 371 Date: Nov. 12, 2024

(30) Foreign Application Priority Data
May 18, 2022 (EP) 22315106.9

(51) Int. Cl. G16B 15/30; G16B 15/20; G16B 40/20

(52) U.S. Cl. CPC G16B 15/30; G16B 15/20; G16B 40/20

(57) ABSTRACT

Methods, systems and apparatus are described for generating a pharmacokinetics (PK) model for predicting a PK value for an antibody of interest. An input dataset is received for a plurality of antibodies. The input dataset comprising data representative of the amino acid sequence of each of said antibodies and an experimentally determined PK value of each of said antibodies. One or more surface properties for each of said antibodies are computed based on the corresponding amino acid sequences. One or more region surface properties for one or more regions of interest are computed for each of said antibodies based on the one or more surface properties computed for each of said antibodies. A grouping from the one or more the regions of interest that produces a maximum correlation between the corresponding computed region surface properties of the grouping and the experimentally determined corresponding PK values is determined. This is used to establish a PK region surface property relationship for the plurality of antibodies. The PK model for predicting the PK value for the antibody of interest is generated based on the PK region surface property relationship. The PK model is configured to receive one or more input region surface properties of said determined grouping for said antibody of interest and output a predicted PK value for said antibody of interest by applying said inputted region surface properties to said relationship.

### CROSS REFERENCE TO RELATED APPLICATIONS

The present application is the national stage entry of International Patent Application No. PCT/EP2023/063077, filed on May 16, 2023, and claims priority to Application No. EP 22315106.9, filed on May 18, 2022, the disclosures of which are incorporated herein by reference.

### TECHNICAL FIELD

This specification relates to methods, systems, and apparatus for predicting pharmacokinetic (PK) values of antibodies prior to in vitro or in vivo analysis.

### BACKGROUND

The introduction of therapeutic monoclonal antibodies (mAb) into the clinical practice revolutionized healthcare to the point of becoming the best-selling drug format over the last few years. The growing popularity of antibody-based therapies has led to the development of next-generation multi-specific antibody therapeutics, which integrate two or more variable regions into a single molecule. Due to their architecture, multi-specific antibodies can engage two or more antigens at once, offering new opportunities to tackle complex diseases compared to the combination of mono-specific antibodies. To date, most of the available information derives from mono-specifics.

Recently, the pharmaceutical industry has shown a great interest in unveiling the pharmacokinetic (PK) principles of antibodies since they provide crucial information regarding the efficacy of drugs and dosing strategies. During an antibody discovery/optimization campaign, candidates with unacceptable PK characteristics are abandoned (or reengineered). Due to budget restrictions and ethical concerns, in vivo testing cannot be conducted for a large panel of candidates. Thus, de-risking before performing in vivo PK evaluation is highly desirable.

### SUMMARY

Correlating in vivo PK data with in vitro properties and in silico descriptors is a desirable objective. There are a range of experimental assays and estimated biophysical properties of mAbs that can be used to predict PK for mono-specific antibodies. These include, for instance, the poly-specificity reagent-binding assay, the affinity-capture self-interaction nanoparticle spectroscopy, the binding to the neonatal Fc receptor (FcRn), and the computational estimation of surface properties of the antibodies. It has been reported that mAbs with excessively positive surface are more frequently internalized into the cells by unspecific pinocytosis and show increased binding affinity to the FcRn, thus preventing the dissociation of the complex. Hence, positive charges can interfere with the IgG-recycling pathway and negatively impact PK. However, as this hypothesis originated from conventional mAbs, its significance to multi-specific formats remains unclear.

Accordingly, there is a desire for an efficient, accurate and rapid mechanism or apparatus for predicting a PK value of any antibody of interest, that will enable the short-listing of the plurality of antibodies that are untested or have unknown PK values. In particular, there is a desire for an efficient, accurate and rapid mechanism or apparatus for predicting a PK value of a multi-specific antibody of interest, that will enable the short-listing of the plurality of multi-specific antibodies that are untested or have unknown PK values.

According to a first aspect of this specification, there is provided a computer-implemented method of generating a pharmacokinetics (PK) model for predicting a PK value for an antibody of interest, the method comprising: receiving an input dataset for a plurality of antibodies, the input dataset comprising data representative of the amino acid sequence of each of said antibodies and an experimentally determined PK value of each of said antibodies; computing one or more surface properties for each of said antibodies based on the corresponding amino acid sequences; computing one or more region surface properties for one or more regions of interest for each of said antibodies based on the one or more surface properties computed for each of said antibodies; determining a grouping from the one or more the regions of interest that produces a maximum correlation between the corresponding computed region surface properties of the grouping and the experimentally determined corresponding PK value to establish a PK region surface property relationship for the plurality of antibodies; and generating the PK model for predicting the PK value for the antibody of interest, wherein the PK model is configured to receive one or more input region surface properties of said determined grouping for said antibody of interest and to output a predicted PK value for said antibody of interest by applying said inputted region surface properties to said PK region surface property relationship.

In some implementations, the PK model may be further configured for computing one or more surface properties for the antibody of interest based on its amino acid sequence; and computing one or more region surface properties for the determined grouping of regions of interest for said antibody of interest based on the one or more surface properties computed for the antibody of interest, thereby providing said input region surface properties.

The computer-implemented method, wherein the generated PK model is further configured for predicting a shortlist of candidate antibodies with desired PK properties for use in in vitro wet lab analysis or for use in in vivo trials.

The computer-implemented method, wherein for each of the candidate antibodies, the method further comprising the steps of: computing one or more surface properties for each of said candidate antibodies based on their corresponding amino acid sequence; and computing one or more region surface properties for the determined grouping from regions of interest for each of said candidate antibodies; inputting data representative of the computed region surface property of each of said candidate antibodies to the generated PK model for predicting a PK value of each of said candidate antibodies; receiving a predicted PK value for each of said candidate antibodies as output from the PK model; and adding a candidate antibody to the shortlist of candidate antibodies when the received predicted PK value of said candidate antibody is indicative of one or more of the desired PK properties defined for the shortlist; and outputting data representative of the shortlist of candidate antibodies for use at least in an in vivo trial or an in vitro wet lab analysis.

The computer-implemented method, wherein computing the one or more surface properties for each of the plurality of antibodies comprises modelling the three-dimensional molecular structure of each of said antibodies and calculating a distribution for said one or more surface properties over the surface of the modelled molecular structure of each of said antibodies.

The computer-implemented method, wherein the computed one or more surface properties comprise one or more of positively charged surface areas, negatively charged surface areas and hydrophobic surface areas.

The computer-implemented method, wherein the surface of the modelled molecular structure of each of said antibodies comprises a plurality of patches of one or more surface properties, each patch having a patch area based on the distribution of said surface property in the modelled molecular structure, wherein the number of patches for each antibody are the same or different for each other antibody of the plurality of antibodies.

The computer-implemented method, wherein the computing of one or more region surface properties for one or more regions of interest for each of said antibodies is based on the area of said patches of one or more surface properties associated with each region of interest.

The computer-implemented method, wherein the plurality of antibodies and the antibody of interest are cross-over dual variable (CODV) antibodies.

The computer-implemented method, wherein at least one region of interest is selected from, complementary domain regions, CDRs, framework regions, or linkers of the variable heavy, VH, or variable light, VL, domains, including CDR1, CDR2, CDR3, FW1, FW2, FW3, FW4 of any of the VH or VL domains of the two variable, V, domains of the CODV antibodies.

The computer-implemented method, wherein the grouping from the regions of interest are CDR1 of VL1, CDR3 of VL and FW1-4 of VL2 and VH2 of the CODV antibodies.

The computer-implemented method, wherein the PK surface property relationship is a linear PK surface property relationship.

According to a second aspect of this specification, there is provided an apparatus comprising a processor, a memory unit and a communication interface, wherein the processor is connected to the memory unit and the communication interface, wherein the processor and memory are configured to implement the computer-implemented method according to any of the features or steps of the first aspect.

According to a third aspect of this specification, there is provided a computer-readable medium comprising data or instruction code, which when executed on a processor, causes the processor to implement the computer-implemented method of any of the features or steps of the first aspect.

According to a fourth aspect, there is provided a system comprising: a three-dimensional surface property module configured for receiving data representative of a plurality of candidate antibodies and computing region surface properties of regions of interest of each of the candidate antibodies; a pharmacokinetic (PK) model module configured for receiving the computed region surface properties corresponding to each of the candidate antibodies for predicting a PK value of said each candidate antibody; a PK comparison module configured for comparing the predicted PK values of the candidate antibodies with desired PK properties and selecting those candidate antibodies of the plurality of candidate antibodies with a PK value meeting said one or more desired PK properties; and an output module configured for outputting a shortlist of candidate antibodies from the selected candidate antibodies based on the comparison for use in in vitro wet lab analysis and/or in vivo trials.

The system may be further configured to implement one or more of the method steps or features according to the first aspect.

According to a fifth aspect of this specification, there is disclosed a non-transitory tangible computer-readable medium comprising data or instruction code for generating a PK model for predicting a PK value for an antibody of interest, which when executed on one or more processors, causes at least one of the processors to perform at least one of the steps of the method of: receiving an input dataset for a plurality of antibodies, the input dataset comprising data representative of the amino acid sequence of each of said antibodies and an experimentally determined PK value of each of said antibodies; computing one or more surface properties for each of said antibodies based on the corresponding amino acid sequences; computing one or more region surface properties for one or more regions of interest for each of said antibodies based on the one or more surface properties computed for each of said antibodies; determining a grouping from the one or more the regions of interest that produces a maximum correlation between the corresponding computed region surface properties of the grouping and the experimentally determined corresponding PK value to establish a PK region surface property relationship for the plurality of antibodies; and generating the PK model for predicting the PK value for the antibody of interest, wherein the PK model is configured to receive one or more input region surface properties of said determined grouping for said antibody of interest and to output a predicted PK value for said antibody of interest by applying said inputted region surface properties to said PK region surface property relationship.

The non-transitory tangible computer-readable medium may be further configured to implement one or more of the method steps or features according to the first aspect.

### BRIEF DESCRIPTION OF THE FIGURES

So that the present disclosure may be more easily understood, embodiments thereof will now be described by way of example only, with reference to the accompanying drawings in which:

FIG. 1a illustrates an example PK model pipeline process according to some embodiments of the present disclosure;

FIG. 1b illustrates an example PK model process according to some embodiments of the present disclosure;

FIG. 1c illustrates an example antibody shortlisting process according to some embodiments of the present disclosure;

FIG. 2a illustrates an example CODV antibody structure and an example three-dimensional (3D model) of a CODV antibody according to some embodiments of the present disclosure;

FIG. 2b illustrates further example CODV antibody structures according to some embodiments of the present disclosure;

FIG. 2c illustrates an example multi-specific CODV Ig-like dataset according to some embodiments of the present disclosure;

FIG. 3a illustrates a system 300 for generating and using a PK model according to some embodiments of the present disclosure;

FIG. 3b illustrates an in silico computational pipeline according to some embodiments of the present disclosure;

FIG. 3c illustrates regions of interest of a CODV antibody structure according to some embodiments of the present disclosure;

FIG. 4a illustrates a set of plots of the correlation between the experimental PK clearance value and the ionic properties in two regions of interest according to some embodiments of the present disclosure;

FIG. 4b illustrates a set of plots of the correlation between the experimental PK clearance values and the hydrophobic properties in regions of interest according to some embodiments of the present disclosure;

FIG. 4c illustrates a cluster plot of the relationships between the plots of FIGS. 4a and 4b according to some embodiments of the present disclosure;

FIG. 4d illustrates a correlation plot of the combined scores (designated as in silico clearance likelihood) of the plots of FIGS. 4a and 4b according to some embodiments of the present disclosure;

FIG. 5 illustrates a plot in relation to two test CODV antibodies in which both test antibodies comprise the same V domains, but their orientation is interchanged, plotted are the positive ionic ratio in CDR1 and CDR3 of VL against the hydrophobic area in FWs of VR2;

FIG. 6 illustrates a plot in relation to two test CODV antibodies in which both test antibodies comprise the same V domains, but their orientation is interchanged, plotted are the positive ionic ratio in CDR1 and CDR3 of VL against the hydrophobic area in FWs of VR2 (left) as well as the combined scores (designated as in silico clearance likelihood) plotted against the experimentally determined in vivo clearance (right);

FIG. 7 illustrates a correlation plot of the combined scores (designated as in silico clearance likelihood) of a validation set of 7 new bi-specific CODV molecules that were first predicted for PK properties using the PK model pipeline of this present disclosure, and later evaluated for in vivo PK (clearance);

FIG. 8 is a schematic illustration of a system/apparatus for performing methods described herein.

Common reference numerals are used throughout the figures to indicate similar features.

### DETAILED DESCRIPTION

Various example implementations described herein relate to method(s), apparatus, and system(s) for automatically, efficiently, and reliably performing in silico predictions or estimates of pharmacokinetic (PK) values of antibodies using a PK model generated from experimental PK data of a set of antibodies. PK values of interest include, without limitation, for example PK clearance or clearance (unit mL/h/kg), PK half-life (T_{1/2}), and PK volume of distribution (V_{SS}) and/or any other suitable PK value of interest. The PK model is generated from a computational pipeline that estimates the surface patch landscape of antibodies (e.g., Cross-Over Dual Variable (CODV) antibodies and/or any other type of antibody), analyses the landscape with experimental PK data available for a set of known antibodies, identifies a grouping of regions of interest over the landscape that maximally correlates with the experimental PK data to form a PK value relationship.

The PK model is formed from the PK value relationship and the identified grouping of the regions of interest. Once formed, the PK model may be used to identify candidate antibodies of interest to generate a short-list of antibodies that meet certain PK value requirements (e.g., fast or slow in vivo clearances) prior to in vitro wet lab analysis and/or in vivo trials of the short-listed antibodies. The surface patch landscape includes a plurality of surface patches (e.g., areas over the surface of the antibody having a particular surface property), each having a surface patch property including, without limitation, for example at least one of ionic properties and hydrophobic properties.

The regions of interest of the antibody may include, without limitation, for example, one or more portions of antigen-binding fragment (Fab) regions, variable binding regions of the Fab; a plurality of portions of variable binding regions associated with variable domains 1 (VR1) or 2 (VR2); a plurality of complementarity-determining regions (CDR) corresponding to each of the VR1 and VR2 domains of the antibody; a plurality of framework (FW) regions corresponding to each of the VR1 and VR2 domains of the antibody; one or more portions of the variable heavy chain (VH) regions and/or variable light chain (VL) binding regions of the VR1 and/or VR2 binding regions of an antibody; one or more portions of the VH1, VH2, VL1 and VL2 binding regions of the VR1 and/or VR2 binding regions of the antibody; a plurality of CDR regions and a plurality of FW regions associated with each of the VH1, VH2, VL1 and VL2 binding regions of the antibody; one or more linkers (L1-L4) of the antibody molecule; and/or any other combination of regions of interest of the antibody as the application demands.

Embodiments of the PK model and/or processes as herein described provide the advantages of an efficient and accurate prediction and estimation of PK values (e.g., PK clearance) in silico rather than performing expensive and laborious in vitro and/or in vivo trials. A further advantage of the PK model as described herein is the reduction in the number of possible candidate antibodies and/or CODV antibodies with unknown PK and the ability to efficiently shortlist the candidate antibodies or CODV antibodies such that only the most promising candidate antibodies or candidate CODV antibodies are selected for in vitro wet lab analysis or screening and/or in vivo trials. Thus, only those antibodies with estimated/predicted PK value/characteristics (e.g., PK clearance) that meet a desired or required PK threshold or characteristic may be shortlisted prior to expensive and/or laborious in vitro wet lab analysis and/or subsequent in vivo trials.

The method of the present application allows for the first time a robust PK prediction of multi-specific, e.g. bi-specific antibodies. Moreover, it succeeds in correlation the size of patches of surface properties with PK values. Regarding CODV antibodies, it established that the orientation of V1 and V2 has an influence on the PK values.

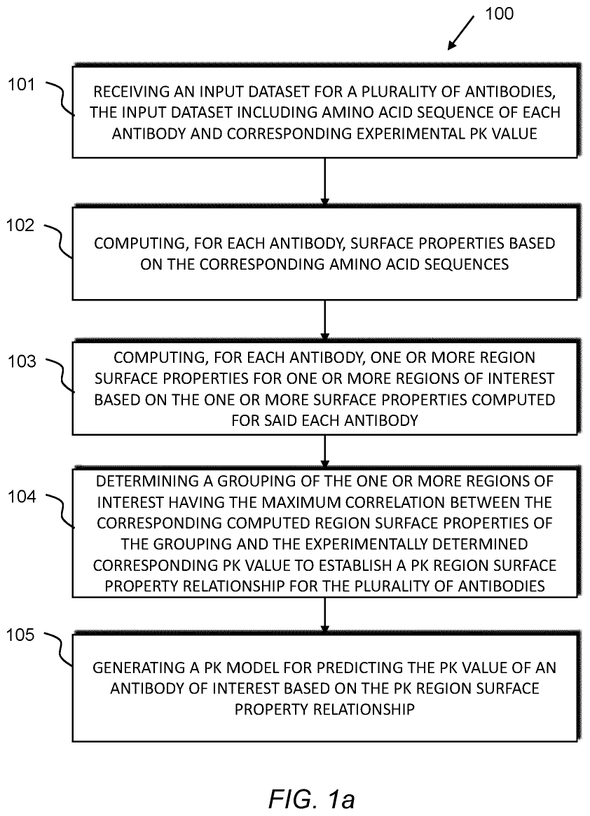

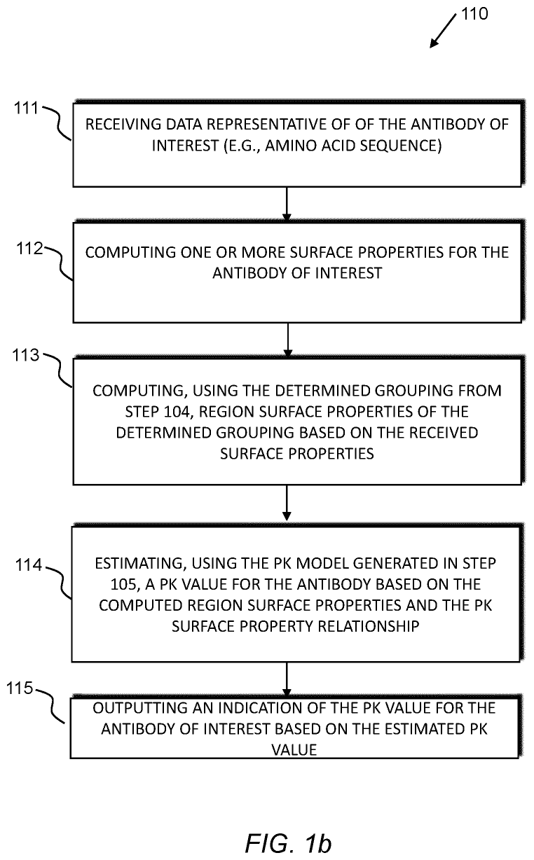

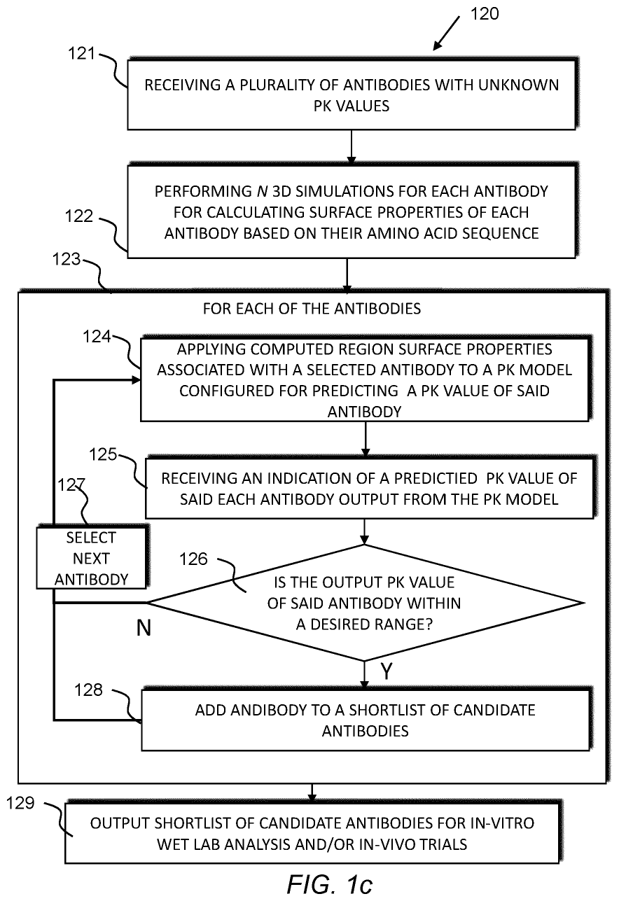

FIG. 1a illustrates an example PK model pipeline process 100 for generating a PK model for predicting PK values for an antibody of interest. The PK model pipeline process 100 includes at least the following steps of: In step 101, receiving an input dataset for a plurality of antibodies, the input dataset including data representative of the amino acid sequence of each of the plurality of antibodies and an experimentally determined PK value (e.g., PK clearance) of said each antibody.

In some embodiments of the present disclosure, the plurality of antibodies and the antibody of interest are multi-specific antibodies.

In some embodiments of the present disclosure, the plurality of antibodies and the antibody of interest are CODV antibodies. In some embodiments of the present disclosure, the plurality of antibodies and the antibody of interest are tetravalent bi-specific CODV antibodies. In some embodiments of the present disclosure, the plurality of antibodies and the antibody of interest are trivalent tri-specific CODV antibodies. In some embodiments of the present disclosure, the plurality of antibodies and the antibody of interest are bivalent bi-specific CODV antibodies.

In step 102, computing one or more surface properties for each of the plurality of antibodies based on their corresponding amino acid sequences.

For example, computing the one or more surface properties for each of the plurality of antibodies may include, without limitation, for example modelling the three-dimensional (3D) molecular structure of each of said antibodies and calculating a distribution for said one or more surface properties over the surface of the 3D modelled molecular structure of each of said antibodies.

In essence, several types of surface properties may be computed for each of the antibodies, which include, without limitation, for example ionic surface properties or hydrophobic surface properties. The ionic surface properties include negatively charged and positively charged surface areas. The hydrophobic surface properties include hydrophobic surface areas. Thus, the computed one or more surface properties of each antibody include surface property areas based on one or more of positively charged surface areas, negatively charged surface areas and hydrophobic surface areas over the surface of the 3D molecular structure of said each antibody. The modelling of 3D structure and the distribution for said one or more surface properties over the surface of the 3D structure can be repeated several times for each of said antibodies. Afterwards, the results of these repetitions can be averaged. In one embodiment, the modelling of 3D structure and the distribution for said one or more surface properties over the surface of the 3D structure are repeated a number of N times (e.g., N=50, N=100, or any other suitable sample size) and averaged afterwards.

The surface of the modelled molecular structure of each of said antibodies includes the one or more surface property areas of one or more surface properties, each surface property area including a plurality of patches of the one or more surface properties. Each patch having a patch area based on the distribution of said surface property in the modelled molecular structure, wherein the number of patches for each antibody are the same or different for each other antibody of the plurality of antibodies. Each patch can be a negatively charged patch; a positively charged patch; or a hydrophobic patch.

In another example, the at least one surface property for a plurality of patches on the surface of each antibody includes an ionic surface property, the ionic surface property of each patch including a positively charged ionic area or a negatively charged ionic area.

In step 103, computing one or more region surface properties for one or more regions of interest for each of said antibodies based on the one or more surface properties computed in step 102 for each of said antibodies.

For example, each region of interest over the surface of the modelled molecular structure of each of said antibodies includes one or more surface areas with one or more surface area properties, each surface property area including one or more patches or a plurality of patches. Computing one or more region surface properties for one or more regions of interest for each of said antibodies is based on the area of said patches of one or more surface properties. Each region of interest may include at least three types of surface properties including, without limitation, for example a negatively charged region, a positively charged region and a hydrophobic region.

In another example, computing at least one region surface property corresponding to one or more regions of interest for each of said antibodies may further include calculating, for each of the antibodies, at least one region surface property of each region of interest based on identifying those areas of said each antibody associated with said each region of interest and combining the corresponding surface property of the identified areas of said each antibody.

In another example, the at least one region surface property corresponding to one or more regions of interest includes an ionic surface property corresponding to the one or more regions of interest. The ionic surface property for each region of interest may include a positive ionic region surface property and a negative ionic region surface property, where computing the region surface property for each of the regions of interest of said each of the antibodies further including calculating the ionic region surface property for each of the regions of interest of said each of the antibodies based on: calculating a positive ionic region surface property for each region of interest of said each of the antibodies by aggregating or averaging those patches identified to be associated with said each region of interest with positively charged ionic areas; and calculating a negative ionic region surface property for each region of interest of said each of said antibodies by aggregating or averaging those patches identified to be associated with said each region of interest with negatively charged ionic areas.

In step 104, determining a grouping from the one or more the regions of interest that produces a maximum correlation between the corresponding computed region surface properties of the grouping and the experimentally determined corresponding PK value. The correlation may be analysed to establish a PK region surface property relationship for the plurality of antibodies.

For example, linear regression may be performed for each grouping from the one or more regions of interest, for all of the plurality of antibodies, based on plotting the data points corresponding computed region surface properties of the grouping for each antibody with the experimentally determined corresponding PK value for said each antibody, and performing a linear regression analysis on the resulting data points. Then the grouping that produces a maximal correlation (e.g., maximal positive or negative correlation) based on the linear regression is selected as the determined grouping. The linear regression output of the selected grouping can be used to establish a PK region surface property relationship for the plurality of antibodies.

Determining a grouping from the one or more regions of interest may further include determining the grouping based on a combination of the one or more regions of interest for said antibodies that produce a maximum correlation (e.g., positive or negative correlation) between the region surface properties of the combination of regions of interest of said antibodies and the corresponding experimentally determined PK values for said antibodies. Establishing a PK surface property relationship can be based on estimating the correlation relationship between the determined grouping and the corresponding experimentally determined PK values for said antibodies. For example, establishing the PK surface property relationship may include calculating a linear PK surface region property relationship from correlating the combined region surface properties of the determined grouping for each of the plurality of antibodies with the corresponding experimentally determined PK values for each of the plurality of antibodies.

Alternatively or additionally, non-linear regression may be performed for each grouping from the one or more regions of interest, for all of the plurality of antibodies, based on plotting the data points corresponding computed region surface properties of the grouping for each antibody with the experimentally determined corresponding PK value for said each antibody, and performing the non-linear regression analysis on the resulting data points. Then the grouping that produces a maximal correlation based on the linear regression is selected as the determined grouping. The linear regression output of the selected grouping is used to establish a PK region surface property relationship for the plurality of antibodies. In any event, a PK region surface property relationship is established.

In step 105, generating the PK model for predicting the PK value for an antibody of interest is based on the PK region surface property relationship of the determined grouping associated with the maximal correlation. The PK model is configured to predict a PK value of an antibody of interest by receiving one or more input region surface properties of said determined grouping for said antibody of interest, processes the input region surface properties of said determined grouping for said antibody of interest according to the PK region surface property relationship, and outputs a predicted PK value for said antibody of interest. The processing may include applying said inputted region surface properties to said PK region surface property relationship.

The PK model may be further configured to receive data representative of the antibody of interest such as, for example, its amino acid sequence and compute one or more surface properties for the antibody of interest based on its amino acid sequence. The PK model may then compute one or more region surface properties for the determined grouping from regions of interest for said antibody of interest based on the one or more surface properties computed for the antibody of interest, thereby providing said input region surface properties.

The PK model may also be configured to assign different PK labels to two or more non-overlapping PK value ranges. The PK model may be further configured to determine which of the two or more PK ranges the predicted PK value belongs and outputting the corresponding PK label and/or the predicted PK value. For example, when the PK value is PK clearance, the labels may correspond to, without limitation, for example “slow”, “medium”, or “fast” PK clearance, or any other PK clearance rate and the like.

As an example, the generated PK model is further configured and used for predicting a shortlist of candidate antibodies with desired PK properties for use in in vitro wet lab analysis and/or in vivo trials. Each candidate antibody is input to the PK model as an antibody of interest, e.g., the amino acid sequence of the candidate antibody is input, where the group region surface properties is computed based on the amino acid sequence of the candidate antibody and applied to the PK region surface property relationship. The candidate antibody exhibits the desired PK properties when the estimated PK value is above or below a certain PK threshold value (depending on what is desired) or the PK value associated with group region surface properties cluster in a PK value region in relation to shortlisted candidate antibodies.

FIG. 1b illustrates an example PK model process 110 for predicting PK values for an antibody of interest. It is assumed that the PK model has been generated in accordance with the PK model pipeline process 100 of FIG. 1a. The PK model process 110 includes the following steps of: In step 11, receiving data representative of the antibody of interest such as, for example, its amino acid sequence.

In step 112, computing one or more surface properties for the antibody of interest based on its amino acid sequence. Computing one or more surface properties for the antibody of interest based on its amino acid sequence may further comprise modelling the 3D molecular structure of said antibody of interest based on its amino acid sequence and calculating a distribution for said one or more surface properties over the surface of the modelled molecular structure of said antibody of interest.

In step 113, computing, using the determined grouping of regions of interest from step 104, one or more region surface properties for said antibody of interest based on the computed one or more surface properties for the antibody of interest. Thereby providing an input of region surface properties for applying to the PK region surface property relationship for estimating the PK value.

In step 114, estimating, using the PK model generated in step 105, a PK value for the antibody of interest based on the computed region surface properties by inputting data representative of the computed region surface property to the generated PK model for predicting a PK value of each of said antibody of interest.

In step 115, outputting an indication of the PK value for the antibody of interest based on the estimated PK value.

FIG. 1c illustrates an example antibody shortlisting process 120 using the PK model process 110 for predicting a candidate shortlist of antibodies with PK values in a desired range or according to desired PK characteristics/properties. It is assumed that the PK model has been generated in accordance with the PK model pipeline process 100 of FIG. 1a. The antibody shortlisting process 120 includes the following steps of:

In step 121, receiving data representative of a plurality of antibodies with unknown PK values. The data representative of said each antibody may include, without limitation, for example the amino acid sequence of said antibody or other standard molecule structure representing said antibody.

In step 122, computing surface properties of each antibody based on their amino acid sequences or other standard molecule structure representing said antibody. For example, the surface properties of each antibody may be computed based on performing a number of N 3D simulations for each antibody for calculating surface properties of said each antibody based on their amino acid sequences or other standard molecule structure representing said antibody. Each 3D simulation for said antibody generating a set of surface properties of said antibody, and aggregating and averaging the N sets of surface properties generated from the 3D simulation for each antibody.

In step 123, processing each antibody of the plurality of antibodies to determine whether the antibody meets the PK criteria/properties for inclusion into the shortlist. Each candidate antibody from the plurality of antibodies is selected for testing/evaluation based on, for each antibody, the following steps of:

In step 124, applying computed region surface properties associated with the selected candidate antibody as the antibody of interest to a PK model (e.g., the PK model output by process 100 or PK model 110 of FIG. 1a or 1b) configured for predicting or estimating a PK value of said antibody.

In step 125, receiving data representative of a predicted or estimated PK value of said antibody of interest output from the PK model.

In step 126, determining whether the output received PK value of said antibody of interest is within the desired range of PK values required for shortlisting said each antibody. If the received PK value is within the desired range of PK values, or reaches above a desired minimum PK threshold value or is below a desired PK maximum threshold value (e.g., Y), then said antibody of interest meets the PK requirements and process 120 proceeds to step 128. If the received PK value is outside the desired range of PK values, or reaches below a desired minimum PK threshold value or is above a desired PK maximum threshold value (e.g., Y), then said antibody of interest does not meet the PK requirements and process 120 proceeds to step 127.

In step 127, the next candidate antibody in the plurality of candidate antibodies is selected as the antibody of interest and the process 120 proceeds to step 124 for testing said selected antibody of interest.

In step 128, adding the antibody of interest as a candidate antibody to the shortlist of candidate antibodies when the received predicted PK value of said candidate antibody is indicative of one or more of the desired PK properties/ranges/thresholds defined for the shortlist.

Once all candidate antibodies in the plurality of antibodies have been processed, outputting data representative of the shortlist of candidate antibodies for use at least in an in vitro wet lab analysis and/or an in vivo trial for analysis of the efficacy and/or PK of said types of antibodies.

Each of the processes 100, 110, 120 of FIGS. 1a-1c may be implemented on an apparatus including a processor, a memory unit, and a communication interface. The processor is connected to the memory unit and the communication interface. The processor and memory may be configured to implement the each of the processes 100, 110, 120 and/or other processes described herein as computer-implemented methods. The processes 100, 110, 120 and/or combinations thereof, modifications thereto based on one or more other processes as herein described may be stored on a computer-readable medium. The computer-readable medium may include data or instruction code, which when executed on a processor, causes the processor to implement one or more of the processes 100, 110, 120 and/or combinations thereof, modifications thereto as one or more computer-implemented methods as described herein.

In some embodiments, the plurality of antibodies and the antibody of interest are cross-over dual variable (CODV) antibodies. However, any type of antibody may be used. It may be preferred that the plurality of antibodies used to generate the PK model are of the same antibody family or same type of antibody. It may be preferred that the antibody format of the plurality of antibodies used to generate the PK model are of the same antibody format. For example, only CODV antibodies are used to generate a CODV PK model, and only monoclonal antibodies (mAb) are used to generate a mAb PK model. For example, only multi-specific antibodies are used to generate a multi-specific PK model and only mono-specific antibodies are used to generate a mono-specific PK model.

“CODV antibodies”, as used herein, comprises all antibodies or antibody fragments which comprise two V domains having a cross-over orientation. CODV antibodies have been previously described in the international patent application WO2012/135345 and WO20161/16626 and the publication Steinmetz et al., MAbs. 2016 July; 8(5):867-78, which are incorporated herein by reference. In some embodiments, the CODV antibody is in CODV-Ig format, e.g., comprises a Fc domain. In some embodiments, the CODV antibody is in CODV-Fc-OL format, e.g., comprises a Fc domain but only one CODV-Fab. In some embodiments, the CODV antibody is in CODV format comprising one CODV-Fab and one conventional Fab, resulting in a tri-specific construct.

CODV antibodies are multi-specific. “Multi-specific”, as used herein, relates to antibodies which specifically bind to more than one target. In some embodiments, the CODV antibodies are bivalent and bi-specific. In some embodiments, the CODV antibodies are trivalent and bi-specific. In some embodiments, the CODV antibodies are trivalent and tri-specific. In some embodiments, the CODV antibodies are tetravalent and bi-specific. In some embodiments, the CODV antibodies are tetravalent and tri-specific. In some embodiments, the CODV antibodies are tetravalent and tetra-specific.

In some embodiments, for CODV antibodies, the regions of interest may be selected from CDR1, CDR2, CDR3, FW1, FW2, FW3, FW4 of any of the VH or VL domains of the two V domains of the CODV antibodies. If may be found that the grouping from the regions of interest that produce the maximal correlation are CDR1 of VL1, CDR3 of VL1 and FW1-4 of VL2 and VH2 of the CODV antibodies.

Although the following description describes the PK model generation pipeline and application in relation to the clearance (or PK clearance) of cross-over dual variable (CODV) Ig-like format antibodies, this is by way of example only and the present disclosure is not so limited, it is to be appreciated by the skilled person that the principles described herein in relation to generating a PK model may be applied to any type of antibody as the application demands.

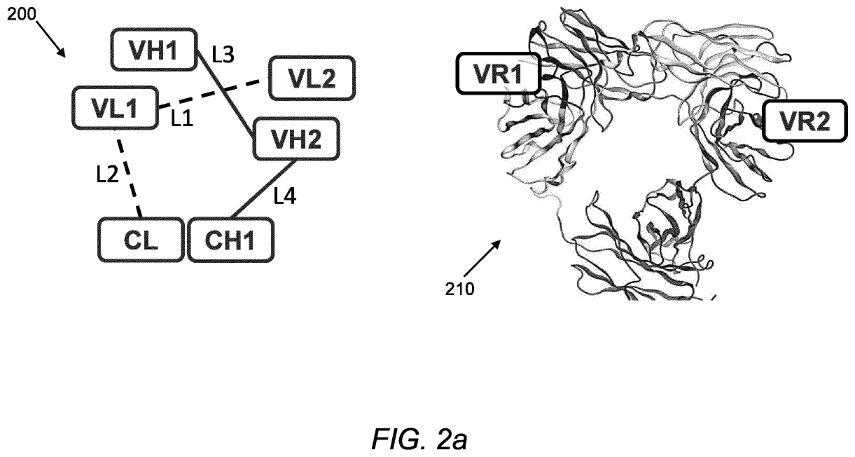

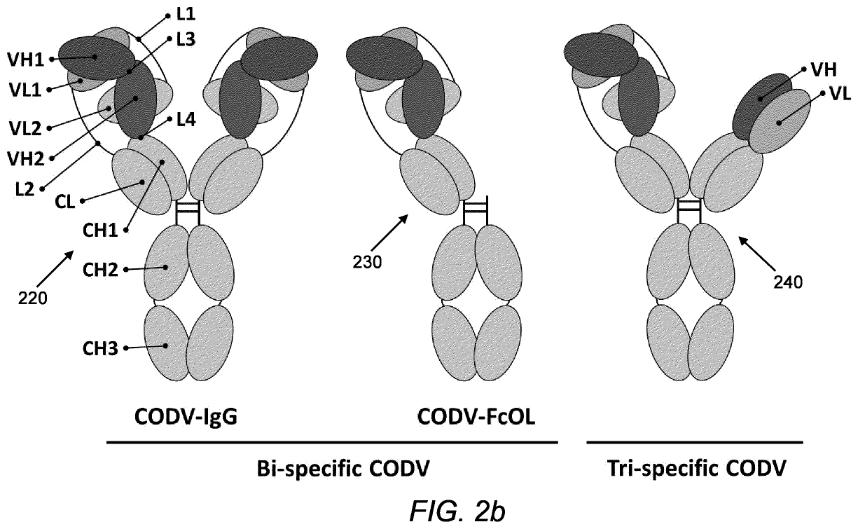

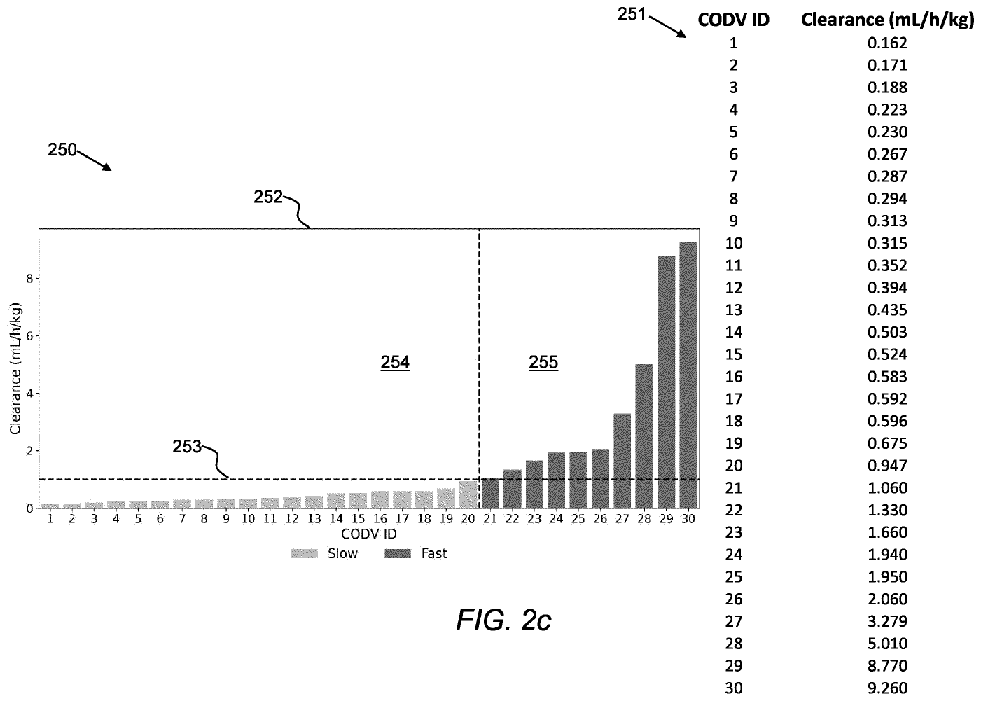

FIGS. 2a and 2b are example CODV antibody architectures/structures 200, 210, 220, 230, and 240 according to some embodiments. The CODV antibody architecture 200 of FIG. 2a is a schematic diagram of a CODV antibody that illustrates two VR binding domains (VR1, VR2) which are linked via linkers (L). VR1 and VR2 are paired together in a cross-over fashion, with VH1 of VR1 bound via L3 to VH2 of VR2 and VL1 of VR1 bound via VL1 to VL2 of VR2. VL1 of VR1 is bound via L2 to the constant domain of the light chain (CL) and VH2 of VR2 is bound via L4 to the first constant domain of the heavy chain (CH1). VH1 pairs with VL1 A (VR1), and VH2 pairs with VL2 (VR2). The CODV antibody architecture 210 is a 3D molecule model of a CODV molecule with VR1 and VR2 regions highlighted generated using Molecular Operating Environment (MOE), (Molecular Operating Environment (MOE), 2020 September Chemical Computing Group ULC, 1010 Sherbooke St. West, Suite #910, Montreal, QC, Canada, H3A 2R7, 2022) or other 3D modelling software and the like.

The CODV antibody architecture 220 of FIG. 2b is another example CODV architecture that includes two VR binding domains paired together in a cross-over fashion as fragment antigen-binding (Fab) arms of an immunoglobulin G (IgG). This allows for the design of bi-specific (bi-Ab) and tri-specific (tri-Ab) modalities shown as CODV antibody architectures 220, 230, and 240. The three modalities 220, 230, and 240 contain at least one CODV-Fab arm and differ from each other in second's arm identity. Regarding the bi-specific modalities, the CODV-IgG has two identical CODV-Fab arms, whereas the CODV-FeOL lacks a second arm and is asymmetrical. The second arm of the displayed CODV tri-specific scaffold is a standard Fab.

In the basic structure of a CODV-Fab arm, the linear sequences of the heavy (HC) and light (LC) chain encode for their respective contributions in the two VR domains of the molecule, named as VR1 and VR2. Each VR targets an epitope of interest, allowing for multi-specificity. Regarding its structure, the CODV-Fab arm is formed by an inner and an outer domain connected by two linkers (L1 and L3) that space the VRs, allowing for the proper folding of the molecule. The constant region of the heavy chain, the VR2 and the linker (L4) that connects them forms the inner domain, while the outer domain is made up of the constant region of the light chain, the VR1 and their linker (L2).

Although several modalities 210-240 are described herein, this is by way of example only and the present disclosure is not so limited, it is to be appreciated by the skilled person that other types of antibody architectures/formats may be applied to the PK pipeline and corresponding PK model generated thereto as the application demands.

FIGS. 2a and 2b are illustrative of different types of modalities that may be used to form a CODV based dataset with known PK values or PK characteristics for generating a PK model, and/or a CODV dataset with unknown PK values or PK characteristics for evaluation and/or screening using the PK model to determine PK characteristics prior to in vitro testing and/or in vivo testing of screened candidate CODV antibodies.

FIG. 2c is illustrates a multi-specific CODV Ig-like dataset 250 comprising a plurality of CODV antibody molecules including 30 different CODV Ig-like antibodies from a diverse selection of target specificities and formats illustrated in CODV dataset table 251. In this example, the CODV dataset consists of 30 CODV antibodies. 23 of the CODV antibodies are bi-specific (including two CODV formats: CODV-IgG and CODV-FcOL) and 7 CODV antibodies are tri-specific (combining one CODV-Fab and one standard Fab arm). These three CODV modalities are illustrated in FIG. 2b.

A CODV clearance table 251 for the 30 CODV molecules designated by CODV ID 1-30 and corresponding known PK clearance values (mL/h/kg) (e.g., PK values). A CODV clearance distribution graph 252 illustrates the distribution of the CODV clearance values of CODV antibodies from CODV clearance table 251. In this example, a PK clearance threshold 253 is set at 1 mL/h/kg, illustrated by a dashed line, may be used in this example to define whether a CODV antibody is in a slow or fast clearance group of CODVs 254 and 255, respectively.

That is, if the PK clearance value for a CODV antibody molecule is above the PK clearance threshold value, then the CODV antibody molecule is considered a fast clearance CODV antibody molecule, or part of the fast clearance group 255. As well, if the PK clearance value for a CODV antibody molecule is at or below the PK clearance threshold value, then the CODV antibody molecule is considered a slow clearance CODV antibody molecule, or part of the slow clearance group 254. It is noted that this PK clearance threshold may be set to any other value and/or multiple PK clearance thresholds may be set to define groupings, multiple clearances, or used to label/characterize the PK clearance of different groups of CODV antibody molecules. Other PK clearance threshold values may be set depending on the type of antibody, for example, from literature for mAbs, PK clearance thresholds in the region of 0.32 mL/h/kg have been cited (Avery et al., MAbs. 2018 February-March; 10(2): 244-255), but this typically depends on the protocols used in determining PK clearance values and approaches may vary with different thresholds. The PK clearance thresholds may be set by a user of the system or defined by the PK value requirements of the in vitro wet lab analysis and/or in vivo trial that a candidate CODV molecule is required to meet.

The CODV dataset 250 of FIG. 2c is used herein as an example CODV dataset to illustrate the pipeline process 100, the resulting PK model process 110, and process 120 for use in selecting candidate PK models with unknown PK values associated with the PK model. Although an example CODV dataset of 20 CODV-like antibody molecules is described herein, this is by way of example only and the present disclosure is not so limited, it is to be appreciated by the skilled person that any antibody dataset with a plurality of antibodies and known PK values (e.g., clearance and/or other PK characteristics) may be used for generating a PK model as described herein and associated for use in predicting or estimating the corresponding PK values of an unknown set of antibodies and/or one or more unknown antibodies and the like.

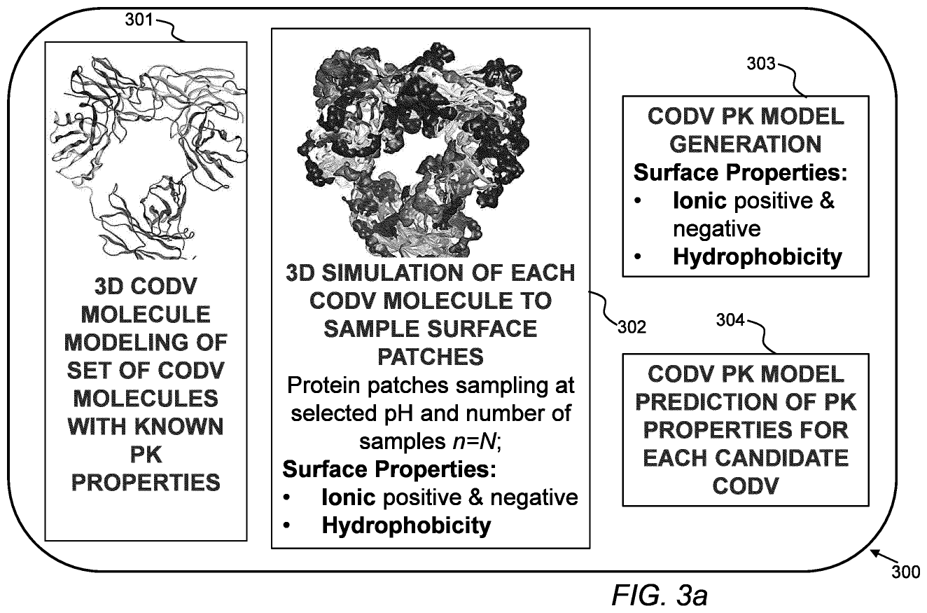

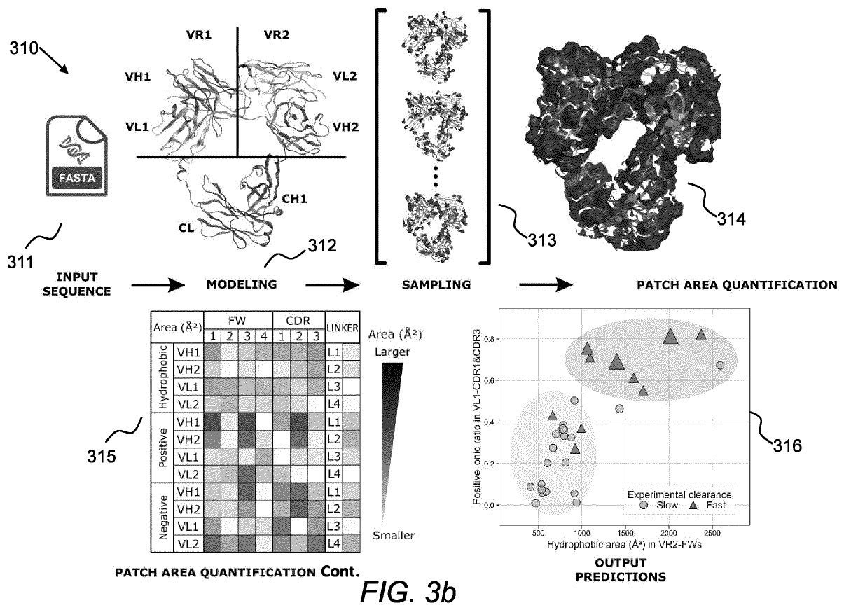

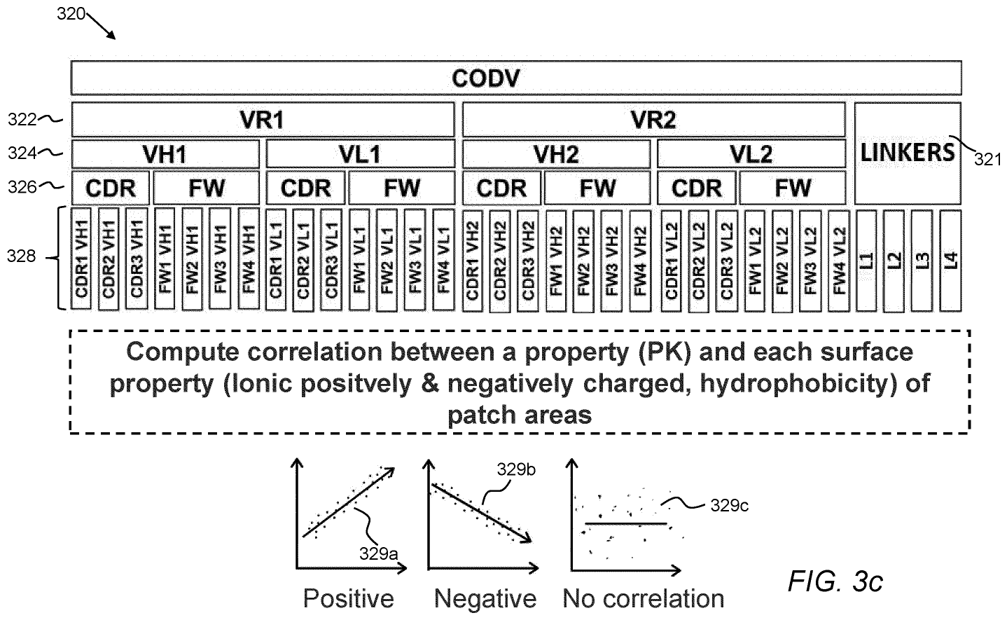

FIG. 3a illustrates a system 300 for generating a PK model for predicting PK values of antibodies. The system 300 including a 3D modelling module 301, a 3D surface property module 302, a PK model generation module 303, and a PK model module 304 connected together. The functionality of 3D modelling module 301, a 3D surface property module 302, a PK model generation module 303, and a PK model module 304 may implement the steps and/or functionality of PK model pipeline process 100, PK model process 110 and antibody shortlisting process 120 of FIGS. 1a, 1b and/or 1c. In operation, the 3D modelling module 301 is configured for modelling the 3D structure of each antibody. For example, modelling the 3D structure of a set of CODV antibody molecules may be based on their amino-acid sequences. The 3D surface property module 302 is configured for receiving data representative of the 3D structure of an antibody and computing region surface properties of regions of interest of each antibody (e.g., steps 101, 102 and 103 of PK model pipeline process 100). The PK model generation module 303 may be configured for generating a PK model based on steps 104 and 105 of the PK model pipeline process 100 of FIG. 1a. The PK model module 304 including one or more the generated PK models configured for receiving the computing region surface properties from the 3D surface property module 302 for an antibody of interest, and applying these to one of the PK models for predicting or estimating a PK value of said antibody of interest. The system 300 may further include an output module for displaying or sending data representative of the predicted or estimated PK value of the antibody of interest to an operator or user of the system 300.

The PK model module 304, may further include functionality (e.g. one or more steps of process 120) for selecting a shortlist of candidate antibodies (e.g., CODV antibodies) from a plurality of antibodies based on desired PK characteristics/properties. In operation, each of the candidate antibodies is input as an antibody of interest to the 3D modelling module 301 for modelling the 3D structure of each antibody of interest. The 3D model of the antibody of interest is provided to the 3D surface property module 302 for computing region surface properties of regions of interest of each antibody of interest. The PK model module 304 receives the computed region surface properties from the 3D surface property module 302 for the antibody of interest, and applies these to one of the PK models for predicting or estimating a PK value of said antibody of interest. The PK model module 304 may further include a PK comparison module (not shown) configured for comparing the predicted or estimated PK values of the candidate antibodies with desired PK properties and selecting those candidate antibodies of the plurality of candidate antibodies with a PK value meeting said one or more desired PK properties. The output module may be configured for outputting a shortlist of candidate antibodies from the selected candidate antibodies based on the comparison for use in in vitro wet lab analysis and/or in vivo trials.

FIG. 3b illustrates an in silico computational pipeline 310 for use in system 300 for generating a PK model for predicting a PK value for an antibody of interest. The computational pipeline 310 is based on the PK model pipeline process 100 with further modifications to steps 101-105 of the PK model pipeline process 100 of FIG. 1a. In this example, the computational pipeline 310 is described with reference to CODV antibodies and the basic CODV-Fab structure. The computational pipeline 310 is configured for generating a PK clearance model for predicting clearance in CODV antibodies. Although PK clearance and a PK clearance model is described herein, this is by way of example only and the present disclosure is not so limited, it is to be appreciated by the skilled person that the computational pipeline 310 and resulting PK model may be applied to any type of antibody and PK, PK value or PK characteristic as the application demands. In this example, it is assumed that a plurality of CODV antibodies having known PK clearance values are used to generate the PK clearance model. For example, the CODV antibody molecule dataset 250 with CODV IDs 1-20 as described with reference to FIG. 2c may be used as the plurality of CODV antibodies with known PK values (e.g., PK clearance in this example).

The computational pipeline 310 may include the following steps of: In step 311, the amino-acid sequence of each of the CODV antibodies from the CODV dataset with known PK values is input to the 3D modelling module 301 in which a 3D modelling system generates a 3D model of each CODV antibody. For example, the amino acid sequence may be input using the FASTA format. Although the FASTA format is described herein, this is by way of example only and the present disclosure is not so limited, it is to be appreciated by the skilled person that any other suitable amino acid sequence data format may be used as the application demands. For example, the software used to generate each 3D model of each CODV may be, without limitation, for example Molecular Operating Environment (MOE), and/or any other suitable 3D modelling system or software as the application demands.

For CODV-Fab structures, the workflow begins with the generation of homology models of the CODV-Fab arms. The homology modeling may be performed based on using MOE. The CDRs, the FWs and the linkers are identified from the sequence using the Chemical Computing Group (CCG) annotation numbering scheme. Although the CCG annotation numbering scheme is described herein, this is by way of example only and the present disclosure is not so limited, it is to be appreciated by the skilled person that any other suitable annotation numbering scheme format may be used as the application demands. Among the five CODV-Fab arms publicly available crystallographic structures (e.g., PDB codes: 608D, 5FHX, 5WHZ, 6O89, and 5HCG), the one with the shortest difference in linker length is used as template. After the selection of the best CODV-Fab arm template, the inner and the outer domain are modelled independently and assembled with the connecting linkers. The VRs are modelled using the best templates from the MOE antibody database. Finally, the whole structure is protonated at a defined pH (e.g., at physiological pH 7.4) and the AMBER10:EHT force field is used to apply a set of minimization protocols (rigid body and free minimizations) to produce a high-quality 3D model.

In step 312, the 3D modelling module 301 may be further configured (or the 3D modelling system) may be configured to model the 3D structure of each CODV antibody based on one or more regions of interest. In this case, the parts or regions of interest of the CODV antibodies that are being modelled are the CH1, VH2, VH1, CL, VL1 and VL2 domains and their connecting linkers (L1, L2, L3, and L4). Further regions of interest are illustrated in FIG. 3c.

In step 313, after the 3D model for each CODV antibody is generated, the 3D model structures of each of the CODV antibodies of the dataset are passed to 3D surface property module 302, in which, for each of the CODV antibodies, the surface side chains (amino acids) of each 3D model of said each CODV antibody are sampled (using MOE) to generate an ensemble of structures aiming to accurately describe the patch surface map of said 3D model structure of said each CODV antibody. In this example, the number of samples N=50 is used such that 50 different sampling structures were modelled for each CODV antibody of the plurality of CODVs with known PK values (e.g., CODVs ID 1-20 of FIG. 2c). Although this example described N=50 samples are used, this is by way of example only and the present disclosure is not so limited, it is to be appreciated by the skilled person that any suitable number of samples N>0 may be used.

The patch surface landscape can be influenced by the conformation of the CODV side-chains, which may adopt multiple energetically favorable orientations. Therefore, using a single structural model to analyze the properties of a molecule's surface might not be entirely representative of reality. Therefore, a set of N=50 different conformers for each CODV-Fab arm model of each CODV antibody in the dataset 250 were generated by using a sampling protocol at a defined pH (e.g., at physiological pH 7.4). Upon generating the conformers, all surface patches are computed. The surface properties of the patches can be hydrophobic, negative, or positively charged, depending on their physicochemical properties.

Each patch has an associated surface's area (measured in Å², or square angstrom) and a list of participating residues. By annotating the CODV-Fab arm sequence using, without limitation, for example the CCG annotation scheme or any other suitable annotation scheme, all patches belonging to each FW, CDR, and linker (16 FWs, 12 CDRs, and 4 linkers for one CODV-Fab arm) were identified. Next, the total hydrophobic, negatively charged, and positively charged areas from all FWs, CDRs, and linkers are obtained for every conformer. Finally, an average value is computed across all conformer structures, obtaining a representative hydrophobic, ionic negatively charged, and ionic positively charged patch area (measured in Å², or square angstrom) from every FW, CDR, and linker over all computed conformations for each CODV antibody of dataset 250. This is performed in steps 314-315 below.

In step 314, the surface patches are computed by 3D surface property module 302 for every sampled structure of said each CODV antibody, where the surface patches include positive charged patches; negative charged patches; or hydrophobic patches. In other words, the surface patches include surface properties including an ionic surface property, or a hydrophobicity surface property, the ionic surface property including ionic positive and ionic negative charges.

In step 315, the 3D surface property module 302 generates a list of surface patches (ionic positive, ionic negative, and hydrophobic), their surface area (measured in Å², or square angstrom), and the residues that are participating in such patches. The surface properties for each of the regions of interest, e.g., a framework (abbreviated FW or FR), a complementarity-determining regions (CDR), a linker or combinations of such regions, are determined by summing/adding the size of the surface properties of those patches that are co-located and/or overlapping with each region of interest.

As an example, where the at least one surface property for a plurality of patches on the surface of each antibody is an ionic surface property, the ionic surface property of each patch includes a positively charged ionic area or a negatively charged ionic area. The ionic surface property for each region of interest includes a positive ionic region surface property and a negative ionic region surface property. Computing the region surface property for each of the regions of interest of said each of the antibodies may further include calculating the ionic region surface property for each of the regions of interest of said each of the antibodies based on: calculating a positive ionic region surface property for each region of interest of said each of the antibodies by aggregating or averaging those patches identified to be associated (e.g. co-located and/or overlapping) with said each region of interest with positively charged ionic areas. Calculating a negative ionic region surface property for each region of interest of said each of said antibodies by aggregating or averaging those patches identified to be associated (co-located and/or overlapping) with said each region of interest with negatively charged ionic areas.

In a further example, at least one surface property for a plurality of patches on the surface of each antibody further includes a hydrophobicity surface property. The hydrophobicity surface property of each patch including an estimated area of hydrophobicity of said each patch. The at least one region surface property for each region of interest further including a hydrophobicity region surface property. Calculating the hydrophobicity region surface property for each of the regions of interest by aggregating or averaging the hydrophobicity surface properties of those patches identified to be associated (e.g., co-located and/or overlapping) with said each region of interest.

In step 316, for each CODV antibody of the plurality of CODV antibodies, the surface properties of the regions of interest may be passed onto the PK model generation module 303. Once the surface properties of the patches have been mapped and combined to surface properties for each of the regions of interest, a plurality of linear correlations for all combinations of the regions of interest between the surface properties of the regions of interest and the experimental PK values (in this case, PK clearance) is performed for the plurality of antibodies in the CODV dataset. The plurality of linear combinations also includes computing additional combinations of surface properties of the regions of interest for different groupings of the regions of interest based on sum/difference/ratio/fraction from the combination of two or more surface properties of different regions of interest.

For example, FIG. 3c illustrates the regions of interest of a CODV antibody structure 320. The regions of interest include, without limitation, for example the linkers 321 (L1, L2, L3, and L4), VR domains 322 including the VR1 and VR2 domains, the VH/VL sub domains 324 including VH1, VL1, VH2, VL2, the CDR/FW 326 for each of the VH/VL domains 324, which include the plurality of regions 328 VH1-CDR1, VH1-CDR2 to VL2-FW3 and FW4-VL2 regions (e.g., there are 28 VH1/VL1/VH2/VL2-CDR1-3/FW1-4 regions). Referring to FIG. 3b, in step 316, the average hydrophobic, ionic negatively charged, and ionic positively charged areas (from, e.g., N=50 3D models) located in the individual regions of interest (e.g., FWs, CDRs and linkers) were used to calculate descriptive scores, or scores, based on averaged properties of the CODV-Fab arm surface. In this regard, the sum and the ratio of many different sets of combinations of regions of interest (e.g., FW and CDR regions 326 and VH1/VL1/VH2/VL2-CDR1-3/FW1-4 regions 328) were performed to find the best grouping of regions of interest. That is, the best grouping of regions of interest for each of the antibodies is the same grouping that results in a maximal correlation (e.g. positive or negative correlation) between the descriptive scoring of that grouping and the experimental PK clearance values of the dataset 250. For example, this may include the ratio of ionic patches (e.g., patches with ionic positive and/or ionic negative surface properties) at a certain location, or total hydrophobic area from a specific region. After performing this task on all possible groupings or combinations of the regions of interest, scores (e.g. averaged surface properties) are generated describing the subdomains of each VR (VH1, VL1, VH2, and VL2) and linkers as well as local subregions of them (a set of CDRs, FWs, or linkers).

The in silico scores were used to perform correlations with the experimental PK clearance dataset 250. For the CODV dataset 250, a strong exponential relationship was observed in the correlations, indicating the experimental PK clearance dataset 250 should be transformed into its natural logarithm (Ln) counterpart to perform linear correlations (positive 329a, negative 329b or no correlation 329c). This may or may not be necessarily applied for other datasets of antibodies and the like. In this example, the Pearson correlation coefficient (r), the Spearman's rank correlation coefficient (rₛ), and the coefficient of determination (r²) were also computed. Although these types of correlation coefficients were computed, this is by way of example only and the present disclosure is not so limited, it is to be appreciated by the skilled person that any suitable type of correlation coefficient may be applied and/or used. The grouping of regions of interest that produced scores or combined scores associated with the maximum correlation or maximum absolute correlation (e.g., maximum positive correlation 329a or maximum negative correlation 329b) are chosen along with the corresponding linear correlation relationship thereof to form the PK model. The relationship associated with the final combined score may be used by the PK model and may be used at part of the PK region surface property relationship of the PK model for predicting or estimating PK clearance values for unknown CODV antibodies associated with the type of CODV antibodies of dataset 250.

In summary, for the CODV dataset 250 of FIG. 2c, the in silico pipeline 310 was used to profile the PK properties of the CODV-Fab arms. The pipeline 310, in steps 311-312 requires the sequence of the CODV-Fab arm to generate a homology model using MOE. The relevant region names in the three-dimensional structure are indicated. Then, in step 313, an ensemble of structures (e.g., N=50) is generated by sampling the side chains of the model, and the surface patches of all conformers are quantified. Next, in steps 314 and 315, the total hydrophobic, positively charged, and negatively charged surface properties for every region of interest such as for every FW and CDR in each domain (VH1, VL1, VH2, and VL2) and each linker and/or combinations thereof are computed (see table in step 315). The intensity of the grey color in the heatmap indicates the size of the total area in for that region of interest (the darker the larger). In step 316, where the plurality of linear correlations (or non-linear correlations) are performed on all combinations of the same type of surface properties of the regions of interest to find a grouping from the regions of interest that has a maximum correlation (positive or negative correlation 329a or 329b) for each type of surface property (e.g., ionic and hydrophobic surface property) with the experimental PK clearance values over all the CODV antibody dataset 250. This includes combining surface properties of the same type such as positive and negative ionic ratios by adding, subtracting, multiplying, and/or dividing in a variety of suitable manners.

Alternatively or additionally, determining a grouping from the regions of interest may include determining an ionic grouping by, for each grouping, combining the corresponding ionic region surface properties (e.g. ionic positive and ionic negative) of each grouping for each of the plurality of antibodies. This may include based on performing the steps of: aggregating or averaging the positive ionic region surface properties of the regions of interest in said each grouping, and aggregating or averaging the negative ionic region surface properties of the regions of interest in said each grouping. From this, determining a combined ionic region surface property of said each grouping based on calculating the ratio of the aggregated positive ionic region surface properties of the grouping with a summation of the aggregated positive ionic region surface properties and the aggregated negative ionic region surface properties of the grouping. Selecting, from all combinations of ionic groupings of the regions of interest, the ionic grouping that produces a maximum correlation (e.g., positive or negative correlation) between the determined combined ionic region surface property of the grouping for each of the plurality of antibodies and the corresponding experimental PK clearance values (or any other PK value) for each of the plurality of antibodies.

Alternatively or additionally, determining a grouping from the regions of interest further including determining a hydrophobicity grouping, for each grouping, by combining the corresponding hydrophobicity region surface properties of each grouping for each of the plurality of antibodies based on aggregating or averaging said corresponding hydrophobicity region surface properties in said each grouping. Selecting, from all combinations of hydrophobicity groupings of the regions of interest, the hydrophobicity grouping that produces a maximum correlation (e.g. positive or negative correlation) between the determined combined hydrophobicity region surface property of the grouping for each of the plurality of antibodies and the corresponding experimentally determined PK values for each of the plurality of antibodies.

The grouping of regions of interest that was found for the CODV dataset 250 that had a maximal correlation with clearance include, for the ionic surface property, the VL1-CDR1&CDR3 regions of interest and, for the hydrophobic surface property, the VR2-FWs regions of interest. Two different types of surface properties were identified to maximally correlate with PK clearance. These included the ionic surface property (e.g., positive and negative ionic charge) based on a positive ionic ratio in VL1-CDR1&CDR3 region and the hydrophobic surface property with a hydrophobic area (Å²) in VR2-FWs region.

For the CODV dataset 250 of FIG. 2c, all the linear correlations for the in silico scores were analysed, in which two different types of surface properties (e.g., ionic and hydrophobic) produced a maximal correlation for a grouping of regions of interest for that type of surface property from all of the different combinations of regions of interest for that type of surface property. These included the positive ionic ratio in VL1-CDR1&CDR3 (Equation 1) and the hydrophobic area in VR2-FWs (Equation 2).

$$Eq . 1 = \frac{Positive area ( {Å}^{2} ) in VL 1 - CDR 1 \& CDR 3}{\begin{matrix}Positive area ( {Å}^{2} ) in VL 1 - CDR 1 \& CDR 3 + \\ Negative area ( {Å}^{2} ) in VL 1 - CDR 1 \& CDR 3\end{matrix}}$$

$$Eq . 2 = Hydrophobic area ( {Å}^{2} ) in VH 2 - FWs + Hydrophobic area ( {Å}^{2} ) in VL 2 - FWs$$

These relationships associated with the ionic score (e.g., Equation 1) and combined hydrophobicity score (e.g., Equation 2) may be used by the PK model and may be used as part of the PK region surface property relationship for predicting or estimating PK clearance values for unknown CODV antibodies associated with the type of CODV antibodies of dataset 250.

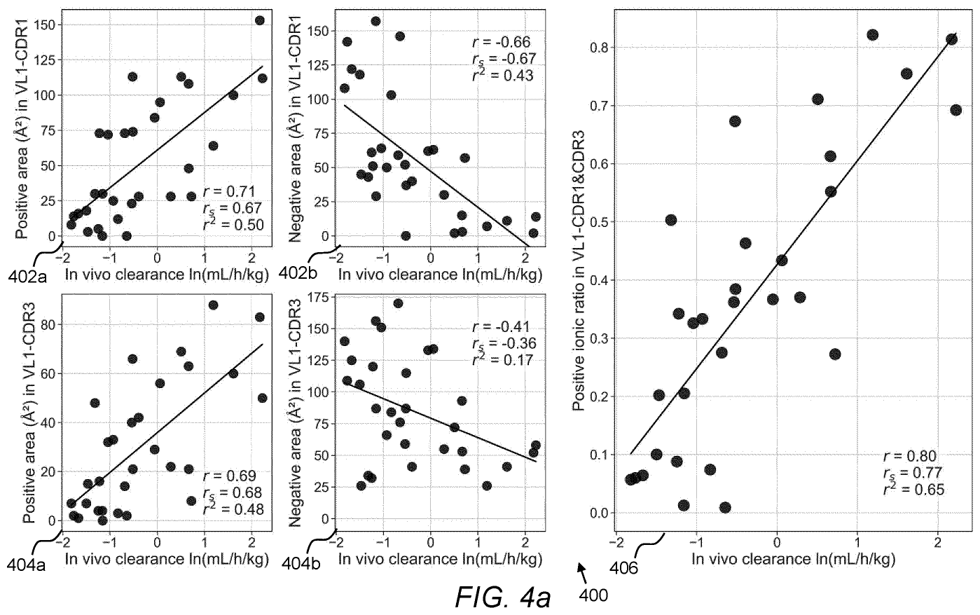

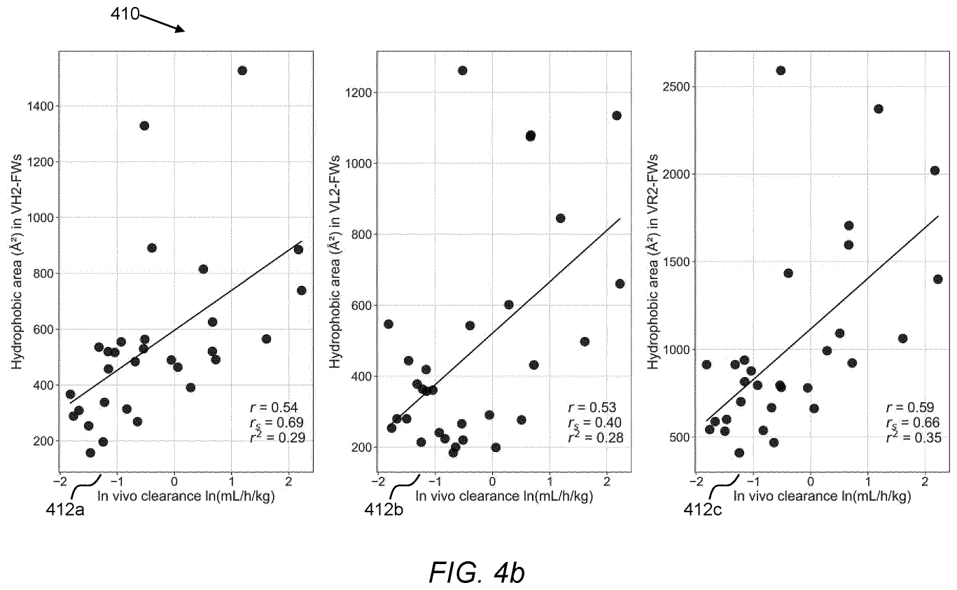

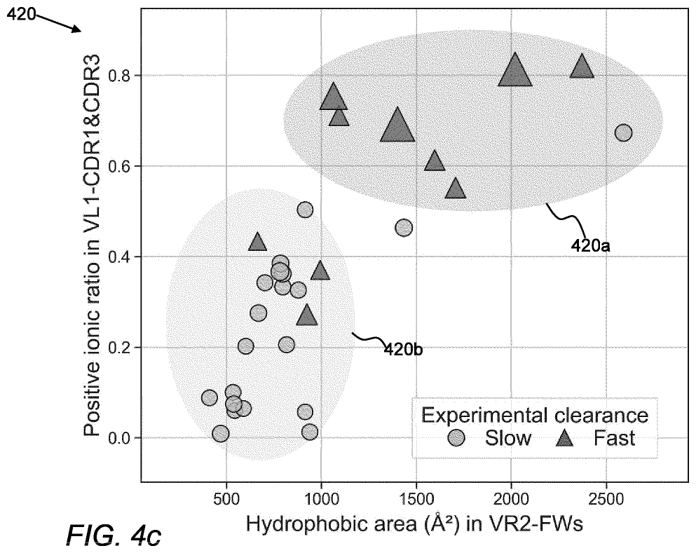

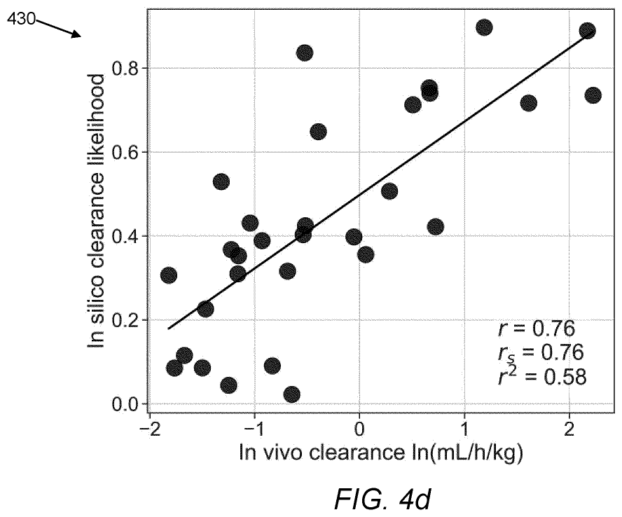

FIG. 4a represents a set of plots 400 of the correlation between the experimental PK clearance value and the ionic properties in two regions of interest, namely, CDR1 and CDR3 of the VL1 domain in the dataset 250 of CODV Ig-like molecules. Plot 402a illustrates the correlation between positive ionic charge area (measured in Å², or square angstrom) for the VL1-CDR1 region and the experimental PK clearance values (In vivo clearance ln(mL/h/kg)) for the antibodies in the dataset 250. Plot 402b illustrates the correlation between negative ionic charge area (measured in Å², or square angstrom) for the VL1-CDR1 region and the experimental PK clearance values (In vivo clearance ln(mL/h/kg)) for the antibodies in the dataset 250. Plot 404a illustrates the correlation between positive ionic charge area (measured in Å², or square angstrom) for the VL1-CDR3 region and the experimental PK clearance values (In vivo clearance ln(mL/h/kg)) for the antibodies in the dataset 250. Plot 402b illustrates the correlation between negative ionic charge area (measured in Å², or square angstrom) for the VL1-CDR3 region and the experimental PK clearance values (In vivo clearance ln(mL/h/kg)) for the antibodies in the dataset 250. As can be seen in plots 402a and 404a, slow PK clearance molecules show smaller positive areas (Å²) in the CDR1 and CDR3 of the VL domain, while fast PK clearance molecules behave differently. The completely opposite behavior was observed for plots 402b and 404b with the negative areas (Å²), indicating that the nature of the ionic properties in the CDRs of the VL1 domain plays an important role in PK clearance. These scores for this region of interest were grouped into a single score, accounting for the positive ionic ratio in VL1-CDR1&CDR3, which is illustrated in plot 406 showing positive ionic ratio vs PK clearance (In vivo clearance ln(mL/h/kg)), where the positive ionic ratio was obtained according to Eq. 1. The r, rₛ, and r² coefficients are displayed in all cases. The best score in terms of correlation coefficients was the positive ionic ratio in the grouping VL1-CDR1&CDR3, thus this relationship can be selected for use in forming the PK region surface property relationship for use in the PK model for predicting or estimating the PK clearance of CODVs with unknown PK clearance values.

FIG. 4b represents a set of plots 410 of the correlation between the experimental PK clearance values and the hydrophobic properties in the regions of interest, namely, the FWs in VH2, VL2, and VR2 in the dataset 250 of CODV Ig-like molecules. Plot 412a illustrates the correlation between hydrophobicity area (measured in Å², or square angstrom) for the VH2-FWs regions and the experimental PK clearance values (In vivo clearance ln(mL/h/kg)) for the antibodies in the dataset 250. Plot 412b illustrates the correlation between hydrophobicity area (measured in Å², or square angstrom) for the VL2-FWs regions and the experimental PK clearance values (In vivo clearance ln(mL/h/kg))) for the antibodies in the dataset 250. Plot 412c illustrates the correlation between hydrophobicity area (measured in Å², or square angstrom) for the VR2-FWs regions and the experimental PK clearance values (In vivo clearance ln(mL/h/kg)) for the antibodies in the dataset 250. These hydrophobic scores for this region of interest may be combined to result in the maximal correlation obtained according to Eq. 2. The r, r₁, and r² coefficients are displayed in all cases. Slow PK clearance molecules show smaller hydrophobic areas (Å²), while fast PK clearance molecules behave differently. The hydrophobic area (Å²) in the grouping VR2-FWs showed the highest correlation coefficients from the three hydrophobic scores.

FIG. 4c represents a cluster plot 420 in which the two best in silico scores from the plot 406 of FIG. 4a and the plot 412c of FIG. 4b were plotted against each other. That is, plot 420 is a graph of the positive ionic ratio in VL1-CDR1&CDR3 (e.g., plot 406 of FIG. 4a) vs the hydrophobic area (Å²) in VR2-FWs (e.g., plot 412c of FIG. 4b). Marker shape indicates slow PK clearance molecules (e.g., circles) and fast PK clearance molecules (e.g., triangle). The size of the marker represents the magnitude of the PK clearance value for that antibody (the larger the marker size, the faster the PK clearance). As can be seen, slow PK clearance molecules tend to cluster together in duster grouping 420b in the lower left corner of the plot 420, while fast PK clearance molecules tend to cluster together in cluster grouping 420a in the upper right region of plot 420.

These two selected in silico scores describe different regions (VR1 and VR2) and physicochemical properties (ionic and hydrophobic) of the CODV-Fab arms that correlate with PK clearance for the dataset 250. After plotting the positive ionic ratio in VL1-CDR1&CDR3 against the hydrophobic area in VR2-FWs in plot 420, two clusters 420a and 420b rich in slow and fast clearance CODV Ig-like molecules were identified. A combined score (designated as “in silico clearance likelihood”) is computed using both in silico scores. The terms “combined score” and “in silico clearance likelihood” are used interchangeably throughout this disclosure. The two in silico descriptors were equally weighted for the construction of the combined score (In silico clearance likelihood), which was obtained according to Equation 3.

$$Eq . 3 = \frac{Positive ionic ratio in VL 1 - CDR 1 \& 3}{2} + \frac{Percentile of hydrophobic area ( {Å}^{2} ) in VR 2 - FWs}{2}$$

Given that one in silico score, the ionic ratio, is a ratio (values between 0 and 1) and the other, the combined hydrophobic averaged properties, is a larger value (ranging between ˜500 Å² to ˜2500 Å²), the scaled percentile of the latter was computed, which will be in the range between 0 and 1.

This facilitates obtaining a combined score representing both ionic and hydrophobic scores at once with the same importance (50% each). The correlation between the combined score and the experimental PK clearance values of dataset 250 is illustrated in FIG. 4d.

FIG. 4d is a plot 430 representing the correlation between the experimental PK clearance values of dataset 250 and the combined score obtained from the combination of the two best in silico scores: the positive ionic ratio in VL1-CDR1&CDR3 and the hydrophobic area (Å²) in VR2-FWs. The combined score was obtained according to Eq. 3. The r, rₛ, and r² coefficients are displayed. Slow PK clearance molecules showed lower combined scores (In silico clearance likelihood) than fast PK clearance molecules. The relationship associated with the combined ionic and hydrophobicity score (e.g., Equation 3) may be used by the PK model as the PK region surface property relationship for predicting or estimating PK clearance values for unknown CODV antibodies associated with the type of CODV antibodies of dataset 250. Thus, in accordance with the processes 100, 110, 120 of FIGS. 1a-1b and the system 300 and pipeline 310 of FIGS. 3a and 3b, a PK model may be generated based on the combined ionic and hydrophobicity score (e.g., Equation 3) and used for predicting or estimating the PK clearance values of unknown CODV antibodies as described with reference to processes 110 and 120 of FIGS. 1b and 1c. The PK models may be used in technical applications such as, without limitation, for example evaluating antibodies with unknown PK values (e.g., clearance) for shortlisting prior to in vitro wet lab analysis and/or in vivo trials.

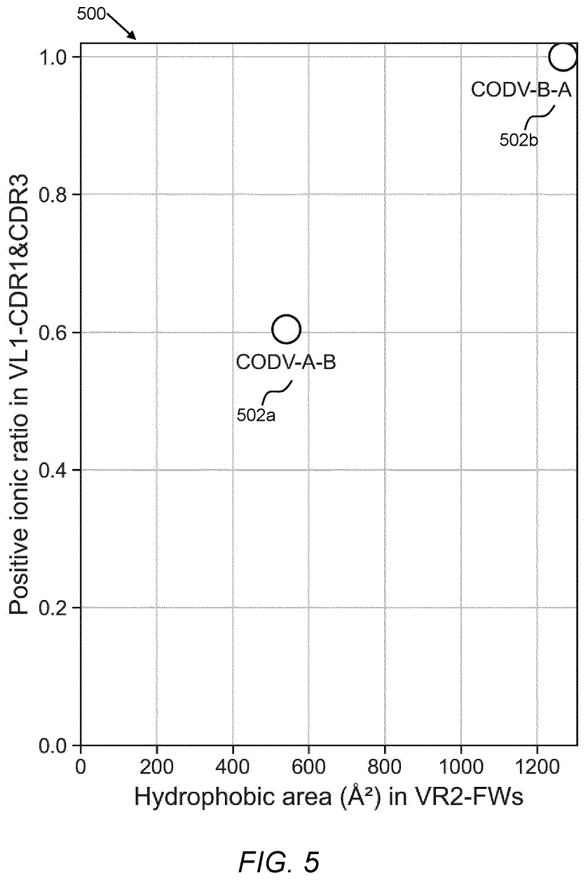

FIG. 5 is a plot 500 of the positive ionic ratio in VL1-CDR1&CDR3 against the hydrophobic area (Å²) in VR2-FWs values of two different test CODV molecules not in the dataset 250. In this case, the two different test CODV molecules have the same binding domains, but in different orientations. The first test CODV molecule (CODV-A-B, 502a) is a CODV comprising the V domains A and B. The second test CODV molecule (CODV-B-A, 502b) is a CODV—comprising the same V domains A and B, but in different orientation compared to CODV-A-B. These were used as test cases as it is known that V domain A has better PK properties (e.g., better PK clearance) than V domain B. Moreover, it is also known that V domain B has larger positive surface areas in their surface (CDRs included) than V domain A. As can be seen, the CODV-A-B with the “good”-V domain A as VL1 domain (e.g., CODV 502a) is the one that shows better in silico surface property parameters, including less positive ionic ratio in VL1-CDR1&CDR3, and less hydrophobicity in VR2-FWs.

By contrast, the CODV-B-A with the “bad” V domain B as VL1 domain (e.g., CODV 502b) is the one that has a very large positive ionic ratio in VL1-CDR1&CDR3 and larger hydrophobic area in VR2-FWs. In addition, in vitro assays were performed for these two CODV antibodies 502a and 502b to predict in vivo clearance based on FcRn chromatography data. FcRn chromatography data describes retention times, which is correlated with PK clearance. Given this, the longer retention times in the FcRn data, the worse the expected clearance pattern or PK clearance values of the antibody. The in vitro FcRn chromatography data of these two CODVs was analysed where it was found that the expected “good” CODV-A-B antibody 502a had a shorter FcRn retention time than the CODV-B-A antibody 502b, which is in agreement with the PK model for CODV molecules.

These results indicate that the PK model should correlate with other future unknown CODVs with unknown clearance values. Thus, there is an alignment between in silico PK models, in vitro FcRn wet lab analysis, and in vivo PK clearance properties. Although in vivo experimental PK clearance values for these CODV antibodies 502a and 502b is not known, the PK model predicts that the CODV 502a has a better PK clearance value than CODV 502b, a result which is also indicated by the FcRn chromatography. This indicates that the PK model, built or formed according to the PK model pipeline processes 100 and 300 of FIGS. 1a and 3a and applied according to processes 110, 120 and as described with reference to FIGS. 3a to 4d, is capable to make accurate predictions for the PK properties of unknown CODV antibodies for shortlisting for in vitro wet lab analysis and/or in vivo trials.

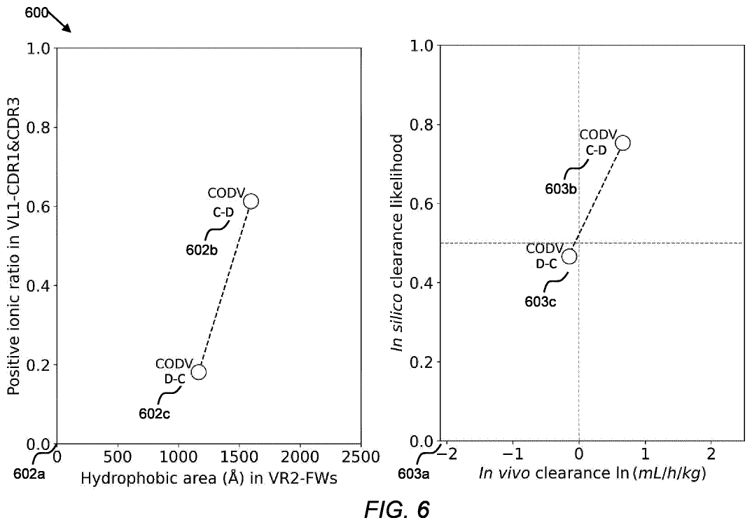

The concept shown in FIG. 5 was experimentally validated by the data shown in FIG. 6. FIG. 6 illustrates two plots: plot 602a and plot 603a. Plot 602a illustrates the positive ionic ratio in VL1-CDR1&CDR3 against the hydrophobic area (Å²) in VR2-FWs values of two different CODV molecules, each comprising the V domains C and D, but in different orientations. The two different orientations of the two CODV molecules are CODV-C-D (602b) and CODV-D-C (602c). It can be seen from plot 602a that the CODV-D-C (602c) antibody is predicted to have better PK clearance values than its counterpart CODV-C-D (602b), as it has lower predicted values on both positive ionic ratio in VL1-CDR1&CDR3 and on the hydrophobic area (Å²) in VR2-FWs.

Plot 603a illustrates the combined score (In silico clearance likelihood) against the experimental clearance (In vivo clearance ln(mL/h/kg)) of the same two different CODV molecules as in plot 602a. It can be seen from plot 603a that the combined score (In silico clearance likelihood) of CODV-D-C (603c) is lower than CODV-C-D (603b), correlating with the experimental clearance (In vivo clearance ln(mL/h/kg)); CODV-D-C (603c) shows slower clearance than CODV-C-D (603b).

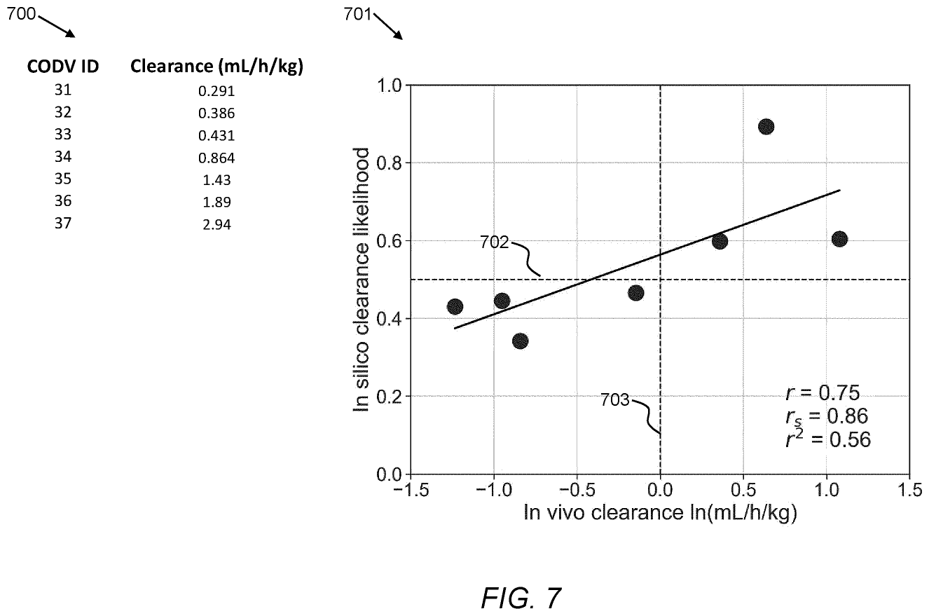

FIG. 7 contains a dataset 700 and a plot 701. Dataset 700 represents a plurality of multi-specific CODV Ig-like antibodies including 7 different antibodies not represented in dataset 250 from a diverse selection of target specificities. The CODV-IgG modality is illustrated in FIG. 2b architecture/structure 220. The plot 701 illustrates the correlation between the in vivo clearance ln(mL/h/kg) and the combined score (In silico clearance likelihood) obtained from Eq. 3. Threshold 702 indicates molecules predicted to show slow clearance profiles (<0.5 units in In silico clearance likelihood) or fast clearance profiles (>0.5 units in In silico clearance likelihood). Threshold 703 indicates molecules with slow experimental clearance (<1 mL/h/kg, or <0 as its natural logarithm counterpart) and fast experimental clearance (>1 mL/h/kg, or >0 as its natural logarithm counterpart). The r, rₛ, and r² coefficients are displayed.

The predicted PK values of the molecules embedded in dataset 700 and illustrated in plot 701 were generated following the PK model pipeline processes 100 and 300 of FIGS. 1a and 3a and applied according to processes 110, 120 and as described with reference to FIGS. 3a to 4d. At the moment of their predictions, there was not available PK data (e.g., half-life, clearance) for any of the 7 molecules embedded in dataset 700. These molecules were selected on purpose from a larger panel of available CODV-IgG solely based on the PK model pipeline of this present disclosure to validate the method with molecules having unknown PK data. This effort aimed to identify 4 molecules with slow clearance profiles and 3 molecules with fast clearance profiles. To determine the in vivo clearance experimentally, the 7 selected CODV-IgG antibodies were expressed in HEK293 cells, two-step purified, and characterized for PK clearance in human FcRn transgenic mouse (Tg32 hFcRn SCID strain) in accordance with Animal Use Protocol (AUP) and institutional animal care and use committee (IACUC) regulations.

Plot 701 shows a very strong correlation between the experimental clearance (In vivo clearance ln(mL/h/kg) and the combined score (In silico clearance likelihood). All 7 molecules represented in dataset 700 and plotted in plot 701 behaved as expected as predicted by the PK model pipeline processes 100 and 300 of FIGS. 1a and 3a and applied according to processes 110, 120 and as described with reference to FIGS. 3a to 4d. The PK model and/or processes as herein described provide the advantage of an efficient and accurate mechanism that substantially reduces the number of possible candidate antibodies and/or CODV antibodies for a shortlist of candidate antibodies or CODV antibodies for wet lab analysis. This then enables a large number of antibodies to be evaluated and screened where only those antibodies with the required estimated/predicted PK value/characteristics (e.g., PK clearance) may be shortlisted prior to expensive and/or laborious in vitro wet lab analysis and/or in vivo trials.

Although the PK model and processes 100, 110, 120 and 310 and system 300 are described with reference to antibodies or CODV antibodies/CODV dataset 250/CODV dataset 700 and the like, this is by way of example only and the present disclosure is not so limited, it is to be appreciated by the skilled person that the PK model and processes 100, 110, 120 and 310 and system 300 as described herein may be applicable to any molecule that may be described in terms of an amino acid sequence such as, without limitation, for example antibodies, CODV antibodies, enzymes, other proteins, and/or any other suitable protein format and the like. Although PK values have been described herein in relation to generating a PK model, this is by way of example only and the present disclosure is not so limited, it is to be appreciated by the skilled person that any other property of interest may be used other than PK values such as, without limitation, for example oligomerization, protein expression, etc., and the like and/or as the application demands.

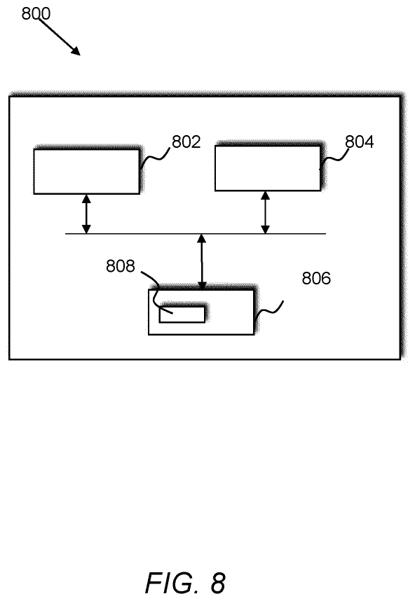

FIG. 8 is a schematic illustration of a system/apparatus for performing methods described herein. The system/apparatus shown is an example of a computing device. It will be appreciated by the skilled person that other types of computing devices/systems may alternatively be used to implement the methods described herein, such as a distributed computing system.

The apparatus (or system) 800 comprises one or more processors 802. The one or more processors control operation of other components of the system/apparatus 800. The one or more processors 802 may, for example, comprise a general-purpose processor. The one or more processors 802 may be a single core device or a multiple core device. The one or more processors 802 may comprise a central processing unit (CPU) or a graphical processing unit (GPU). Alternatively, the one or more processors 802 may comprise specialised processing hardware, for instance a RISC processor or programmable hardware with embedded firmware. Multiple processors may be included.

The system/apparatus comprises a working or volatile memory 804. The one or more processors may access the volatile memory 804 in order to process data and may control the storage of data in memory. The volatile memory 804 may comprise RAM of any type, for example Static RAM (SRAM), Dynamic RAM (DRAM), or it may comprise Flash memory, such as an SD-Card.

The system/apparatus comprises a non-volatile memory 806. The non-volatile memory 806 stores a set of operation instructions 808 for controlling the operation of the processors 802 in the form of computer readable instructions. The non-volatile memory 806 may be a memory of any kind such as a Read Only Memory (ROM), a Flash memory or a magnetic drive memory.

The one or more processors 802 are configured to execute operating instructions 808 to cause the system/apparatus to perform any of the methods or processes described herein with reference to FIGS. 1a to 7. The operating instructions 808 may comprise code (e.g., drivers) relating to the hardware components of the system/apparatus 800, as well as code relating to the basic operation of the system/apparatus 800. Generally speaking, the one or more processors 802 execute one or more instructions of the operating instructions 808, which are stored permanently or semi-permanently in the non-volatile memory 806, using the volatile memory 804 to temporarily store data generated during execution of said operating instructions 808.

Implementations of the methods described herein may be realised as in digital electronic circuitry, integrated circuitry, specially designed ASICs (application specific integrated circuits), computer hardware, firmware, software, and/or combinations thereof. These may include computer program products (such as software stored on e.g., magnetic discs, optical disks, memory, Programmable Logic Devices) comprising computer readable instructions that, when executed by a computer, such as that described in relation to FIG. 8, cause the computer to perform one or more of the methods described herein.

Any system feature as described herein may also be provided as a method feature, and vice versa. As used herein, means plus function features may be expressed alternatively in terms of their corresponding structure. In particular, method aspects may be applied to system aspects, and vice versa.

Furthermore, any, some and/or all features in one aspect can be applied to any, some and/or all features in any other aspect, in any appropriate combination. It should also be appreciated that particular combinations of the various features described and defined in any aspects of the present disclosure can be implemented and/or supplied and/or used independently.

Although several embodiments have been shown and described, it would be appreciated by those skilled in the art that changes may be made in these embodiments without departing from the principles of this disclosure, the scope of which is defined in the claims.

1. A computer-implemented method of generating a pharmacokinetics (PK) model for predicting a PK value for an antibody of interest, the method comprising:
receiving an input dataset for a plurality of antibodies, the input dataset comprising data representative of an amino acid sequence of each of said antibodies and an experimentally determined PK value of each of said antibodies;
computing one or more surface properties for each of said antibodies based on the corresponding amino acid sequences;
computing one or more region surface properties for one or more regions of interest for each of said antibodies based on the one or more surface properties computed for each of said antibodies;
determining a grouping from the one or more regions of interest that produces a maximum correlation between the corresponding computed region surface properties of the grouping and the experimentally determined corresponding PK value to establish a PK region surface property relationship for the plurality of antibodies; and
generating the PK model for predicting the PK value for the antibody of interest, wherein the PK model is configured to receive one or more input region surface properties of said determined grouping for said antibody of interest and to output a predicted PK value for said antibody of interest by applying said inputted region surface properties to said PK region surface property relationship.

2. The computer-implemented method of claim 1, wherein the PK model is further configured to:
compute one or more surface properties for the antibody of interest based on an amino acid sequence of the antibody of interest; and
compute one or more region surface properties for the determined grouping of regions of interest for the antibody of interest, based on the one or more surface properties computed for the antibody of interest, wherein the computed surface region properties are used as the input region surface properties for the PK model.

3. The computer-implemented method of claim 1, wherein the generated PK model is further configured for predicting a shortlist of candidate antibodies with desired PK properties for use in in vitro wet lab analysis, or for use in in vivo trials, or both.

4. The computer-implemented method of claim 3, wherein for each of the candidate antibodies, the method further comprises:
computing one or more surface properties for each of said candidate antibodies based on its corresponding amino acid sequence;
computing one or more region surface properties for the determined grouping of regions of interest for each of said candidate antibodies;
inputting data representative of the computed region surface property of each of said candidate antibodies to the generated PK model for predicting a PK value of each of said candidate antibodies;
receiving a predicted PK value for each of said candidate antibodies as output from the PK model; and
adding a candidate antibody to the shortlist of candidate antibodies when the received predicted PK value of said candidate antibody is indicative of one or more of the desired PK properties defined for the shortlist; and
outputting data representative of the shortlist of candidate antibodies for use at least in an in vitro wet lab analysis or an in vivo trial.

5. The computer-implemented method of claim 1, wherein computing the one or more surface properties for each of the plurality of antibodies comprises modelling a three-dimensional molecular structure of each of said antibodies and calculating a distribution for said one or more surface properties over the surface of the modelled molecular structure of each of said antibodies.

6. The computer-implemented method of claim 1, wherein the computed one or more surface properties comprise one or more of: positively charged surface areas, negatively charged surface areas, or hydrophobic surface areas.

7. The computer-implemented method of claim 5, wherein the surface of the modelled molecular structure of each of said antibodies comprises a plurality of patches of one or more surface properties, each patch having a patch area based on the distribution of said surface property in the modelled molecular structure, wherein a number of patches for each antibody are the same or different for each other antibody of the plurality of antibodies.

8. The computer-implemented method of claim 7, wherein the computing of one or more region surface properties for one or more regions of interest for each of said antibodies is based on the area of said patches of one or more surface properties associated with each region of interest.

9. The computer-implemented method of claim 1, wherein the plurality of antibodies and the antibody of interest are cross-over dual variable, CODV, antibodies.

10. The computer-implemented method of claim 9, wherein at least one region of interest is selected from, complementary domain regions, CDRs, framework regions, or linkers of the variable heavy, VH, or variable light, VL, domains, including CDR1, CDR2, CDR3, FW1, FW2, FW3, FW4 of any of the VH or VL domains of two variable, V, domains of the CODV antibodies.

11. The computer-implemented method of claim 10, wherein the grouping from the regions of interest are CDR1 of VL1, CDR3 of VL1 and FW1-4 of VL2 and VH2 of the CODV antibodies.

12. The computer-implemented method of claim 1, wherein the PK surface property relationship is a linear PK surface property relationship.

13. The computer-implemented method of claim 1, wherein determining a grouping from the one or more regions of interest that produces maximum correlation between the corresponding computed region surface properties of the grouping and the experimentally determined corresponding PK value comprises;
computing a combined score from the corresponding computed region surface properties of the grouping and determining a maximum correlation between said combined score and the experimentally determined corresponding PK value to establish the PK region surface property relationship for the plurality of antibodies.

14. The computer-implemented method of claim 13, wherein the combined score comprises two equally weighted computed region surface properties.

15. A system comprising one or more processors, one or more memory units and a communication interface, wherein the one or more processors are connected to the one or more memory units and the communication interface, and wherein the one or more memory units store instructions that, when executed by the one or more processors, cause the one or more processors to perform operations for generating a PK model for predicting a PK value for an antibody of interest, the operations comprising:
receiving an input dataset for a plurality of antibodies, the input dataset comprising data representative of an amino acid sequence of each of said antibodies and an experimentally determined PK value of each of said antibodies;
computing one or more surface properties for each of said antibodies based on the corresponding amino acid sequences;
computing one or more region surface properties for one or more regions of interest for each of said antibodies based on the one or more surface properties computed for each of said antibodies;
determining a grouping from the one or more regions of interest that produces a maximum correlation between the corresponding computed region surface properties of the grouping and the experimentally determined corresponding PK value to establish a PK region surface property relationship for the plurality of antibodies; and
generating the PK model for predicting the PK value for the antibody of interest, wherein the PK model is configured to receive one or more input region surface properties of said determined grouping for said antibody of interest and to output a predicted PK value for said antibody of interest by applying said inputted region surface properties to said PK region surface property relationship.

16. One or more non-transitory computer-readable storage media storing instructions which when executed on one or more processors cause the one or more processors to perform operations for generating a PK model for predicting a PK value for an antibody of interest, the operations comprising:
receiving an input dataset for a plurality of antibodies, the input dataset comprising data representative of an amino acid sequence of each of said antibodies and an experimentally determined PK value of each of said antibodies;
computing one or more surface properties for each of said antibodies based on the corresponding amino acid sequences;
computing one or more region surface properties for one or more regions of interest for each of said antibodies based on the one or more surface properties computed for each of said antibodies;
determining a grouping from the one or more regions of interest that produces a maximum correlation between the corresponding computed region surface properties of the grouping and the experimentally determined corresponding PK value to establish a PK region surface property relationship for the plurality of antibodies; and
generating the PK model for predicting the PK value for the antibody of interest, wherein the PK model is configured to receive one or more input region surface properties of said determined grouping for said antibody of interest and to output a predicted PK value for said antibody of interest by applying said inputted region surface properties to said PK region surface property relationship.

17. (canceled)

18. The one or more non-transitory computer-readable storage media of claim 16, wherein the PK model is further configured to:
compute one or more surface properties for the antibody of interest based on an amino acid sequence of the antibody of interest; and
compute one or more region surface properties for the determined grouping of regions of interest for the antibody of interest, based on the one or more surface properties computed for the antibody of interest, wherein the computed surface region properties are used as the input region surface properties for the PK model.

19. The one or more non-transitory computer-readable storage media of claim 16, wherein the generated PK model is further configured for predicting a shortlist of candidate antibodies with desired PK properties for use in in vitro wet lab analysis, or for use in in vivo trials, or both.

20. The one or more non-transitory computer-readable storage media of claim 19, wherein for each of the candidate antibodies, the operations further comprise:
computing one or more surface properties for each of said candidate antibodies based on its corresponding amino acid sequence;
computing one or more region surface properties for the determined grouping of regions of interest for each of said candidate antibodies;
inputting data representative of the computed region surface property of each of said candidate antibodies to the generated PK model for predicting a PK value of each of said candidate antibodies;
receiving a predicted PK value for each of said candidate antibodies as output from the PK model; and
adding a candidate antibody to the shortlist of candidate antibodies when the received predicted PK value of said candidate antibody is indicative of one or more of the desired PK properties defined for the shortlist; and
outputting data representative of the shortlist of candidate antibodies for use at least in an in vitro wet lab analysis or an in vivo trial.

21. The one or more non-transitory computer-readable storage media of claim 16, wherein computing the one or more surface properties for each of the plurality of antibodies comprises modelling a three-dimensional molecular structure of each of said antibodies and calculating a distribution for said one or more surface properties over the surface of the modelled molecular structure of each of said antibodies.
